(19) United States
(12) Patent Application Publication
(10) Pub. No.: US 2011/0105393 A1
(43) Pub. Date: May 5, 2011

(54) METHODS FOR PROMOTING STEM CELL PROLIFERATION AND SURVIVAL

(76) Inventors: Andreas Androutsellis-Theotokis, Athens (GR); Ronald D.G. McKay, Bethesda, MD (US)

(21) Appl. No.: 12/673,576

(22) PCT Filed: Aug. 14, 2008
(86) PCT No.: PCT/US08/73204

§ 371 Date: Feb. 15, 2010

Related U.S. Application Data
(60) Provisional application No. 60/965,094, filed on Aug. 16, 2007.

(51) Int. Cl. A61K 38/28; C12N 5/071; C12N 5/0797; A61K 38/18; A61P 25/28; A61P 25/16; A61P 25/00

(52) U.S. Cl. 514/6.5; 435/375; 514/7.6; 514/9.1

(57) ABSTRACT

Methods are disclosed herein for increasing the number of stem cells or precursor cells. The number of stem cells can be increased by increasing survival and/or cell proliferation of the cells. The methods include contacting the cells with an effective amount of a Notch ligand, an effective amount of a growth factor, and an effective amount of angiopoietin-2 (Ang-2). In several embodiments, the methods include contacting the cells with an effective amount of a Jak inhibitor. In several non-limiting examples, the growth factor is insulin or glial derived neurotrophic factor (GDNF), or a combination thereof. In additional non-limiting examples, the Notch ligand is Delta. The cells can be in vivo or in vitro. Methods are also disclosed here for the treatment of a neurodegenerative disorder or spinal cord injury in a subject. In several non-limiting examples, the subject has Parkinson's disease or Alzheimer's disease.

### CROSS REFERENCE TO RELATED APPLICATION

This claims the benefit of U.S. Provisional Application No. 60/965,094, filed on Aug. 16, 2007, which is incorporated herein by reference in its entirety. The subject matter of this application is related to the subject matter of PCT Application No. PCT/US2006/034988, filed Sep. 7, 2006, which claims the benefit of U.S. Provisional Application No. 60/715,935, filed Sep. 8, 2005. These applications are incorporated by reference herein in their entirety.

### FIELD

This application relates to the field of stem cells, such as neuronal stem cells, specifically to methods and agents that are of use to promote stem cell proliferation and survival.

### BACKGROUND

Neurodegenerative disorders encompass a range of seriously debilitating conditions including Parkinson's disease, amyotrophic lateral sclerosis (ALS, “Lou Gehrig's disease”), multiple sclerosis, Huntington's disease, Alzheimer's disease, Pantothenate kinase associated neurodegeneration (PKAN, formerly Hallervorden-Spatz syndrome), multiple system atrophy, diabetic retinopathy, multi-infarct dementia, macular degeneration, and the like. These conditions are characterized by a gradual but relentless worsening of the patient's condition over time. These disorders affect a large population of humans, especially older adults.

Parkinson's disease is a degenerative disorder of the central nervous system that often impairs the sufferer's motor skills and speech. Parkinson's disease is widespread, with a prevalence estimated between 100 and 250 cases per 100,000 in North America. Parkinson's disease is characterized by muscle rigidity, tremor, a slowing of physical movement (bradykinesia) and, in extreme cases, a loss of physical movement (akinesia). The primary symptoms are the results of decreased stimulation of the motor cortex by the basal ganglia, normally caused by the insufficient formation and action of dopamine, which is produced in the dopaminergic neurons of the brain. Secondary symptoms may include high level cognitive dysfunction and subtle language problems. Parkinson's disease is both chronic and progressive. The symptoms of Parkinson's disease result from the loss of pigmented dopamine-secreting (dopaminergic) cells, secreted by the same cells, in the pars compacta region of the substantia nigra (literally “black substance”). These neurons project to the striatum and their loss leads to alterations in the activity of the neural circuits within the basal ganglia that regulate movement.

At present, there is no cure for Parkinson's disease, but medications or surgery are used to provide relief from the symptoms. The most widely used form of treatment is levadopa (L-DOPA) in various forms. L-DOPA is transformed into dopamine in the dopaminergic neurons by L-aromatic amino acid decarboxylase (often known by its former name dopa-decarboxylase). However, only 1-5% of L-DOPA enters the dopaminergic neurons. The remaining L-DOPA is often metabolised to dopamine elsewhere, causing a wide variety of side effects. Carbidopa and benserazide are dopa decarboxylase inhibitors that are used to prevent the metabolism of L-DOPA before it reaches the dopaminergic neurons and are generally given as combination preparations of carbidopa/levodopa (co-careldopa) and benserazide/levodopa (co-beneldopa). The dopamine-agonists bromocriptine, pergolide, pramipexole, ropinirole, cabergoline, apomorphine and lisuride are moderately effective but have side effects including somnolence, hallucinations and for insomnia. Inhibitors of monoamine oxidase-B (MAO-B) inhibit the breakdown of dopamine secreted by the dopaminergic neurons reduce the symptoms. However, these drugs have additional side effects such as insomnia. In addition, the use of L-DOPA in conjunction with an MAO-B inhibitor results in increased mortality rates. Thus, there clearly is a need for additional modalities to treat neurodegenerative diseases, such as Parkinson's.

### SUMMARY

Methods are disclosed herein for increasing the number of stem cells or precursor cells. The number of stem cells can be increased by increasing survival and/or proliferation of the cells. The methods include contacting the cells with an effective amount of a Notch ligand, an effective amount of a growth factor, and an effective amount of angiopoietin-2 (Ang-2). The cells can be in vivo or in vitro. In several embodiments, the methods include contacting the cells with an effective amount of a Janus activated kinase (Jak) inhibitor. In additional embodiments, the growth factor is insulin, glial derived neurotrophic factor (GDNF), or a combination thereof. In further embodiments, the Notch ligand is Delta.

Methods are also disclosed herein for the treatment of a neurodegenerative disorder or spinal cord injury in a subject. The methods include administering to the subject a therapeutically effective amount of a Notch ligand, a therapeutically effective amount of a growth factor and a therapeutically effective amount of Ang-2. Optionally, a therapeutically effective amount of a Jak inhibitor can also be administered to the subject. In several embodiments, the growth factor is insulin, GDNF, or a combination thereof. In further embodiments, the Notch ligand is Delta. In additional embodiments, the subject has Parkinson's disease, Alzheimer's disease or a spinal cord injury.

The foregoing and other features and advantages will become more apparent from the following detailed description of several embodiments, which proceeds with reference to the accompanying figures.

### BRIEF DESCRIPTION OF THE FIGURES

FIGS. 1a-1c are a set of digital images and bar graphs showing vascular signals expand neuronal stem cells (NSCs) in vitro. FIG. 1a is a bar graph showing Dll4 and insulin co-operatively promote the generation of new cells in adult NSC cultures. In contrast, adult NSC cultures from Hairy enhancer of split (Hes) 3 null mice display a marked reduction in response to Delta (Dll) 4 and insulin. FIG. 1b is a digital image showing that following insulin starvation (2-d), acute (1-h) insulin treatment induces DAPT-sensitive FIG. 1c is a digital image showing that cleavage of Notch and phosphorylation of insulin-like growth factor (IGF) 1/insulin receptor. Insulin starvation (16-h) reduces the phosphorylation of STAT3 on serine 727; subsequent addition of insulin (1-h) reinstates it.

FIGS. 2a-2b are digital images and a bar graph showing vascular signals expand NSCs in vitro. FIG. 2a is a set of digital images showing that Ang-1 (0.5 μg/ml) induces time-dependent Akt phosphorylation and inhibits mammalian target of rapamycin (mTOR) phosphorylation; Ang-2 (0.5 μg/ml) induces STAT3 phosphorylation on serine 727. FIG. 2b is a bar graph showing that Ang-2 (0.5 μg/ml, 5-d) promotes the generation of adult subventricular zone (SVZ) NSCs in the presence of Dll4 (size bar, 100 μm).

FIGS. 3a-3b are a series of bar graphs showing the activation of the NSC niche in vivo. FIG. 3a is a bar graph showing single intra-ventricular injections of various pharmacological treatments promote the generation of new cells throughout the brain and spinal cord within 5-days. FIG. 3b is a bar graph showing single intra-ventricular injections of various pharmacological treatments promote the generation of Hes3+ cells throughout the brain parenchyma, associated with blood vessels within 5-days.

FIGS. 4a-4b are a diagram and a bar graph showing activation of the NSC niche in the ventral midbrain. FIG. 4a is a diagram showing a single intra-ventricular injection of the mix increases the circumference of the aqueduct that harbours TH+ cells (at 5-d). FIG. 4b is a bar graph showing that a single intra-ventricular injection of various treatments increases the number of Hes3+ cells around the aqueduct. (All size bars, 50 μm).

FIGS. 5a-5c are bar graphs showing that short-term imaging techniques predict long-term functional recovery. FIG. 5a is a bar graph showing that T2 signal (water) from magnetic resonance imaging (MRI) of rats 1-day following 6OHDA treatment correlates with behavioural recovery (T2 low vs. high cut-off volume chosen: 75 mm²). FIG. 5b is a bar graph showing that the T1 signal (manganese) from MRI imaging of rats 1-d to 50-d following 6OHDA treatment correlates with behavioural recovery (T1 low vs. high cut-off signal chosen: 1100 ms). FIG. 5c is a set of bar graphs showing different treatments (administered 2-weeks following 6OHDA lesion) induce different vascular effects; quantitation of small and large (>20 μm diameter) vessels.

FIGS. 6a-6e are a set of graphs showing that treatments that activate the NSC niche confer neuroprotection of dopaminergic cells and behavioural recovery. FIG. 6a is a graph showing single intra-ventricular injections of Dll4 or mix following 6-OHDA lesion in adult rats promote long-lasting behavioural recovery as assessed by rotometry measurements following amphetamine treatment. FIG. 6b is a graph showing T1 MRI signal correlates with behavioural (rotometry) improvement in control and treated, 6OHDA-lesioned rats. FIG. 6c is a bar graph showing single intra-ventricular injection of the mix 2-weeks following 6-OHDA lesion rescues TH+ processes in the striatum and TH+ cell bodies in the substantia nigra. FIG. 6d is a bar graph showing various treatments 2-weeks following 6-OHDA lesion rescue TH+/Fluorogold+ cells in the substantia nigra (Fluorogold was given 1-week prior to perfusion, ipsilateral to the lesion site). FIG. 6e is a bar graph showing treatment with insulin, Ang-2 and the mix, but not with Dll4, 2-weeks following 6OHDA lesion induce an increase in the cell body diameter of TH+/Fluorogold+ cells in the substantia nigra (ipsilateral to the 6OHDA lesion).

FIG. 7 is a schematic diagram of signal transduction model in isolated NSC cultures as disclosed herein. Homogenous NSC cultures express vascular cytokines and receptors that regulate their expansion in an autocrine and paracrine manner. STAT3-Ser727 is a critical component of NSC survival, downstream of mTOR and Akt. Akt is activated by insulin (added in the culture medium) and Notch cleavage (mediated by cell-to-cell contact). Hes3 is downstream of STAT3 and induces expression of the mitogen Shh. Shh also regulates the expression of Ang-1 and Ang-2. Ang-1 activates the Tie-2 receptor which contributes to cell expansion by activating Akt, and opposes it at the same time by activating p38 MAP kinase (which indirectly inhibits mTOR function). Ang-2 is a partial antagonist of Ang-1 and results in increased mTOR and STAT3-Ser727 phosphorylation, without affecting Akt phosphorylation. Thus, the net effect of insulin, Dll4, and Ang-2 is NSC expansion.

FIG. 8 is a schematic diagram of a signal transduction model in the neurovascular niche as disclosed herein. In the neurovascular niche, NSCs (Hes3+) are spaced regularly along blood vessels. Vascular endothelial Dll4 activates Notch receptors on NSCs to promote their survival by indirect phosphorylation on STAT3-Ser727, a critical regulator of NSC survival. STAT3-Ser727 induces Shh expression which promotes the survival of adjacent neurons. When NSCs differentiate, they express GDNF which also promotes neuronal survival. Thus, a NSC can promote neuronal survival during its self-renewal state, and during differentiation. NSCs also respond to Ang-1 secreted by pericytes that coat vascular endothelial cells. Ang-1 activates Akt to promote survival but suppresses STAT3-Ser727 and thus induces quiescence. Vascular endothelial cells antagonize the effects of Ang-1 on the NSCs by secreting Ang-2; however, this process is more pronounced during injury- or cancer-dependent angiogenesis. Thus, injury and cancer, in addition to stimulating angiogenesis, also activate the NSC niche. Because Hes3+ NSCs are spaced regularly along blood vessels and because this signalling model utilizes secreted, diffusible factors, the NSC activation signal propagates along the external surface of the vasculature in a wave-like fashion. This mechanism would ensure fast activation of the NSC niche over long distances.

FIG. 9 is a schematic representation of the neurovascular niche.

FIGS. 10a-d are a set of digital images and bar graphs showing neural precursors specifically express and respond to the Tie-2 receptor. FIG. 10a is a digital image of a Western blot analysis showing that fetal neural cells in culture express Tie-2 and that Ang2 treatment induces the time-dependent phosphorylation of this receptor. FIG. 10b is a bar graph showing Ang2 induces the expansion of neural precursor cultures from the adult rat SVZ. FIG. 10c is a schematic representation of the areas dissected from the adult rat brain (subventricular zone (SVZ), Lateral: ˜bregma 1.5 to −0.36 mm. FIG. 10d is a bar graph showing that growth factor treatment also induces the expansion of neural precursor cultures from an area of the adult rat outside the established germinal zones. The culture medium contained Ang2 and an inhibitor of the Jak kinase (CT: Combined treatment).

FIG. 11 is a three dimensional diagrammatic representation of confocal optical sections and a bar graph showing angiogenic factors activate neural precursors in vivo. Single intracerebroventricular injections of Dll4, Ang2 or CT increased the number of Hes3+ cells in the adult striatum and substantia nigra within 5 days.

FIG. 12 is a pair of bar graphs showing combinations of pro- and anti-angiogenic factors maintain normal vascular density. Vascular density (“object number”) and total vessel surface area (“object area”) was quantitated by pattern recognition software in striatal sections stained for RECA-1.

FIGS. 13a-c are a series of graphs showing that injured dopamine neurons are protected from death by single treatments with angiogenic factors. FIG. 13a is a graph showing that single intracerebroventricular injections of various treatments 2 weeks after 6-hydroxydopamine (6OHDA) lesion in adult rats promote long-lasting behavioral recovery assessed by amphetamine-induced rotometry. FIG. 13b is a bar graph illustrating that treatment with Dll4, Ang2, and CT result in an increase in tyrosine hydroxylase (TH)+ signal in the lesioned striatum (data are expressed as % ipsi- vs. contra-lateral TH optical density; contralateral TH signal was similar in all treatment groups). FIG. 13c is a bar graph showing that dopamine neurons were retrogradely labeled by fluorogold injections in the striatum, 1-week prior to sacrifice; data are expressed as % increases in Fluorogold+ cell bodies in the substantia nigra; all fluorogold-labeled cell bodies in the substantia nigra expressed TH.

### DETAILED DESCRIPTION

Methods are disclosed herein for increasing the number of stem cells or precursor cells, either in vitro or in vivo. The number of stem cells can be increased by increasing survival and/or proliferation of the cells. The methods include contacting the cells with an effective amount of a Notch ligand, an effective amount of a growth factor, and an effective amount of angiopoietin-2 (Ang-2).

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### TERMS

In order to facilitate review of the various embodiments of this disclosure, the following explanations of specific terms are provided:

Agent: Any polypeptide, compound, small molecule, organic compound, salt, polynucleotide, or other molecule of interest.

Akt (protein kinase B): A serine/threonine kinase that is an enzyme involved in signal transduction pathways in cell proliferation, apoptosis, angiogenesis, and diabetes. In mammals three isoforms of Akt (a, b, g or Akt 1, 2, 3) have been described. These isoforms exhibit a high degree of homology, but differ slightly in the localization of their regulatory phosphorylation sites. Akta is the predominant isoform in most tissues, whereas the highest expression of Aktb is observed in the insulin-responsive tissues, and Aktg is abundant in brain tissue. Each Akt isoform is composed of three functionally distinct regions: an N-terminal pleckstrin homology (PH) domain that provides a lipid-binding module to direct Akt to phosphatidylinositol (PIP)₂ and PIP₃, a central catalytic domain, and a C-terminal hydrophobic motif.

Akt is constitutively phosphorylated at Ser¹²⁴, in the region between the PH and catalytic domains, and on Thr⁴⁵⁰, in the C-terminal region (in Akta, the most widely studied isoform) in unstimulated cells. Activation of Akt involves growth factor binding to a receptor tyrosine kinase and activation of PI 3-K, which phosphorylates membrane bound PIP₂ to generate PIP₃. The binding of PIP₃ to the PH domain anchors Akt to the plasma membrane and allows its phosphorylation and activation by PDK1. Akt is fully activated following its phosphorylation at two regulatory residues, a threonine residue on the kinase domain and a serine residue on the hydrophobic motif, which are structurally and functionally conserved within the AGC kinase family. Phosphorylation at Thr³⁰⁸ and Ser⁴⁷³ is required for the activation of Akta, while phosphorylation at Thr³⁰⁹ and Ser⁴⁷⁴ activates Aktb. Phosphorylation at Thr³⁰⁵ activates Aktg. Phosphorylation of a threonine residue on the kinase domain, catalyzed by PDK1, is essential for Akt activation. It causes a charge-induced conformational change, allowing substrate binding and increased rate of catalysis. Akt activity is augmented about 10-fold by phosphorylation at the serine residue by PDK2.

Alter: A change in an effective amount of a substance of interest, such as a polynucleotide or polypeptide. The amount of the substance can be changed by a difference in the amount of the substance produced, by a difference in the amount of the substance that has a desired function, or by a difference in the activation of the substance. The change can be an increase or a decrease. The alteration can be in vivo or in vitro. In several embodiments, altering an effective amount of a polypeptide or polynucleotide is at least about a 50%, 60%, 70%, 80%, 90%, 95%, 96%, 97%, 98%, 99%, or 100% increase or decrease in the effective amount (level) of a substance, the proliferation and/or survival of a cells, or the activity of a protein, such as an enzyme. In another embodiment, an alteration in polypeptide or polynucleotide or enzymatic activity affects a physiological property of a cell, such as the differentiation, proliferation, or senescence of the cell.

Angiopoietin-2 (Ang-2): An antagonist for angiopoietin-1 and its receptor, Tie2. Human wild-type Ang-2 is 496 amino acids in length and is expressed by endothelial cells at sites of vascular remodeling. Production of Ang-2 has been implicated in tumor development. Ang-2 binds to TIE2 receptor and counteracts blood vessel maturation/stability mediated by angiopoietin-1. Without being bound by theory, in the absence of angiogenic inducers, such as vascular endothelial growth factor, (VEGF), Ang-2-mediated loosening of cell-matrix contacts induces endothelial cell apoptosis with consequent vascular regression. In concert with VEGF, Ang-2 facilitates endothelial cell migration and proliferation, thus serving as a permissive angiogenic signal.

An exemplary amino acid sequence for human Ang-2 is shown in GENBANK® Accession No. NP_{—}001138, Jul. 30, 2007, and GENBANK® Accession No. BAA95590, May 10, 2000, which are incorporated herein by reference. The sequences of several additional mammalian Ang-2 proteins are known, including, but no limited to the rat and mouse sequences (see GENBANK® Accession Nos. O35462, Jul. 10, 2007 and GENBANK® Accession No. NP_{—}031452, Jul. 30, 2007, incorporated herein by reference).

Suitable biologically active variants can be Ang-2 analogs or derivatives. By “analog” is intended an analog of either Ang-2 or an Ang-2 fragment that includes a native Ang-2 sequence and structure having one or more amino acid substitutions, insertions, or deletions. Analogs having one or more peptoid sequences (peptide mimic sequences) are also included (see International Patent Publication No. WO 91/04282). By “derivative” is intended any suitable modification of Ang-2 or a fragment or analog, such as glycosylation, phosphorylation, or other addition of foreign moieties, as long as the Ang-2 activity is retained. Methods for making Ang-2 fragments, analogs and derivatives are available in the art. In addition to the above-described Ang-2, the methods disclosed herein can also employ an active mutant or variant thereof. The term “active Ang-2” includes mutated forms of the naturally occurring Ang-2. Ang-2 variants will generally have at least 70%, preferably 80%, more preferably 85%, even more preferably 90% to 95% or more, and for example 98% or more amino acid sequence identity to the amino acid sequence of the reference Ang-2 molecule. A mutant or variant may, for example, differ by as few as 1 to 10 amino acid residues, such as 6-10, as few as 5, as few as 4, 3, 2, or even 1 amino acid residue.

The sequence identity can be determined as described herein. For Ang-2, one method for determining sequence identify employs the Smith-Waterman homology search algorithm (Meth. Mol. Biol. 70:173-187, 1997) as implemented in MSPRCH program (Oxford Molecular) using an affine gap search with the following search parameters: gap open penalty of 12, and gap extension penalty of 1. In one embodiment, the mutations are “conservative amino acid substitutions” using L-amino acids, wherein one amino acid is replaced by another biologically similar amino acid. Conservative amino acid substitutions are those that preserve the general charge, hydrophobicity, hydrophilicity, and/or steric bulk of the amino acid being substituted.

One skilled in the art, using art known techniques, is able to make one or more point mutations in the DNA encoding Ang-2 to obtain expression of an Ang-2 polypeptide mutant (or fragment mutant) having an activity for use in methods disclosed herein. Standard techniques for site directed mutagenesis, are known in the art (see, for example, Gilman et al., Gene 8:81, 1979 or Roberts et al., Nature 328:731, 1987) to introduce one or more point mutations into the cDNA that encodes the Ang-2.

Animal: Living multi-cellular vertebrate organisms, a category that includes, for example, mammals and birds. The term mammal includes both human and non-human mammals. Similarly, the term “subject” includes both human and veterinary subjects.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Central Nervous System (CNS): The part of the nervous system of an animal that contains a high concentration of cell bodies and synapses and is the main site of integration of nervous activity. In higher animals, the CNS generally refers to the brain and spinal cord.

Ciliary Neurotropic Factor (CNTF): An acidic cytosolic protein of approximately 24 kDa. CNTF does not display any homology to other neurotropic factors. At the protein level CNTF from rabbits and humans show approximately 76 percent sequence identity. Rat CNTF and human CNTF show 84 percent homology.

CNTF is found predominantly in peripheral nerve tissues. The main source appears to be myelin-associated Schwann cells in peripheral nerves and astrocytes in the central nervous system. CNTF appears to be expressed relatively late during ontogenesis. CNTF has been proposed to be a lesion factor that is released after nerve injuries and that, in combination with other factors, promotes the survival and the regeneration of neurons. In vitro CNTF promotes the growth of parasympathetic neurons and sympathetic, sensory, and spinal motor neurons.

Degenerate variant: A polynucleotide encoding a polypeptide, such as Ang-2, that includes a sequence that is degenerate as a result of the genetic code. There are 20 natural amino acids, most of which are specified by more than one codon. Therefore, all degenerate nucleotide sequences are included in the invention as long as the amino acid sequence of the polypeptide encoded by the nucleotide sequence is unchanged.

Differentiation: Refers to the process whereby relatively unspecialized cells (such as embryonic stem cells or other stem cells) acquire specialized structural and/or functional features characteristic of mature cells. Similarly, “differentiate” refers to this process. Typically, during differentiation, cellular structure alters and tissue-specific proteins appear.

Effective amount or Therapeutically effective amount: The amount of agent sufficient to prevent, treat, reduce and/or ameliorate the symptoms and/or underlying causes of any of a disorder or disease, or to increase the number of cells, such as to increase the survival and/or proliferation of cells. In one embodiment, an “effective amount” is sufficient to reduce or eliminate a symptom of a disease. In another embodiment, an effective amount is an amount sufficient to overcome the disease itself. In a further example, an effective amount of an agent is an amount that produces a statistically significant increase in the number of cells in culture as compared to a control, such as a culture not treated with the agent or treated with vehicle alone.

Expand: A process by which the number or amount of cells in a cell culture is increased due to cell division. Similarly, the terms “expansion” or “expanded” refers to this process. The terms “proliferate,” “proliferation” or “proliferated” may be used interchangeably with the words “expand,” “expansion”, or “expanded.” Typically, during an expansion phase, the cells do not differentiate to form mature cells, but divide to form more cells.

Fibroblast growth factor (FGF): Any suitable fibroblast growth factor, derived from any animal, and functional fragments thereof. A variety of FGFs are known and include, but are not limited to, FGF-1 (acidic fibroblast growth factor), FGF-2 (basic fibroblast growth factor, bFGF), FGF-3 (int-2), FGF-4 (hst/K-FGF), FGF-5, FGF-6, FGF-7, FGF-8, FGF-9 and FGF-98. “FGF” refers to a fibroblast growth factor protein such as FGF-1, FGF-2, FGF-4, FGF-6, FGF-8, FGF-9 or FGF-98, or a biologically active fragment or mutant thereof. The FGF can be from any animal species. In one embodiment, the FGF is mammalian FGF, including but not limited to, rodent, avian, canine, bovine, porcine, equine and human. The amino acid sequences and method for making many of the FGFs are well known in the art.

The amino acid sequence of human FGF-1 and a method for its recombinant expression are disclosed in U.S. Pat. No. 5,604,293. The amino acid sequence of human FGF-2 and methods for its recombinant expression are disclosed in U.S. Pat. No. 5,439,818, herein incorporated by reference. The amino acid sequence of bovine FGF-2 and various methods for its recombinant expression are disclosed in U.S. Pat. No. 5,155,214, also herein incorporated by reference. When the 146 residue forms are compared, their amino acid sequences are nearly identical, with only two residues that differ.

The amino acid sequence of FGF-3 (Dickson et al., Nature 326:833, 1987) and human FGF-4 (Yoshida et al., PHAS USA 84:7305-7309, 1987) are known. When the amino acid sequences of human FGF-4, FGF-1, FGF-2 and murine FGF-3 are compared, residues 72-204 of human FGF-4 have 43% homology to human FGF-2; residues 79-204 have 38% homology to human FGF-1; and residues 72-174 have 40% homology to murine FGF-3. The cDNA and deduced amino acid sequences for human FGF-5 (Zhan et al., Molec. and Cell. Biol. 8(8):3487-3495, 1988), human FGF-6 (Coulier et al., Oncogene 6:1437-1444, 1991), human FGF-7 (Miyamoto et al., Mol. and Cell. Biol. 13(7):4251-4259, 1993) are also known. The cDNA and deduced amino acid sequence of murine FGRF-8 (Tanaka et al., PNAS USA 89:8928-8932, 1992), human and murine FGF-9 (Santos-Ocamp et al., J. Biol. Chem. 271(3):1726-1731, 1996) and human FGF-98 (provisional patent application Ser. No. 60/083,553, which is hereby incorporated herein by reference in its entirety) are also known.

FGF-2 (also known as bFGF or bFGF-2), and other FGFs, can be made as described in U.S. Pat. No. 5,155,214. The recombinant bFGF-2, and other FGFs, can be purified to pharmaceutical quality (98% or greater purity) using the techniques described in detail in U.S. Pat. No. 4,956,455.

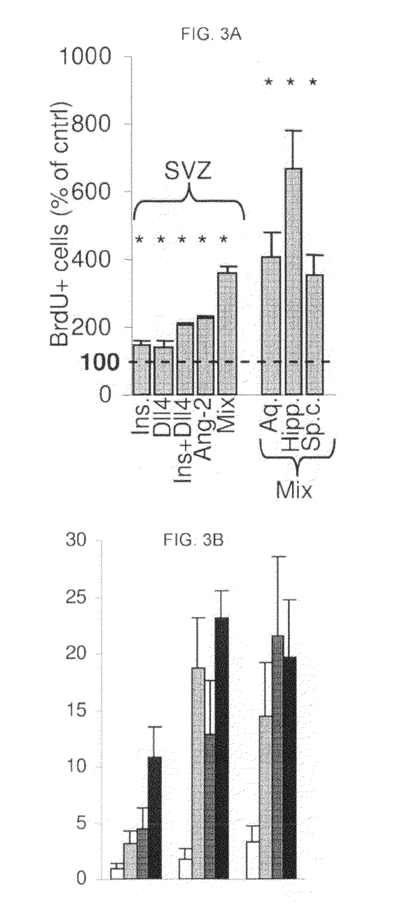

FGF-4 is the product of the hst oncogene (also known as hst-1 or hst). The amino acid sequence for human FGF-4 was first disclosed by Yoshida et al., Proc. Natl. Acad. Sci. USA 84:7305-7309, 1987, at FIG. 3. The endogenous human protein encoded has a molecular mass of 23 kDa. FGF-4 has been implicated recently as one of the molecules that directs outgrowth and patterning of the limb during chick embryonic growth (see Adelaide et al., Oncogene 2:413-416, 1988; see also U.S. Pat. No. 6,277,820).

Fibroblast growth factor-8 (FGF-8), alternatively known as androgen-induced growth factor (AIGF) is a member of the FGF family known to influence embryogenesis and morphogenesis. The in situ embryonic expression pattern suggests a unique role of FGF-8 in mouse development, especially in gastrulation, brain development, and limb and facial morphogenesis (Ohuchi et al., Biochem. Biophys. Res. Commun. 204(2):882-888, 1994). Northern blot expression reveals a unique temporal and spatial pattern of FGF-8 expression in the developing mouse and suggests a role for this FGF in multiple regions of ectodermal differentiation in the post-gastrulation mouse embryo (Heikinheimo et al., Mech. Dev. 48(2):129-138, 1994). A sequence of FGF-8 is shown in U.S. Pat. No. 6,277,820.

Biologically active variants of FGF are also of use with the methods disclosed herein. Such variants should retain FGF activities, particularly the ability to bind to FGF receptor sites. FGF activity may be measured using standard FGF bioassays, which are known to those of skill in the art. Representative assays include known radioreceptor assays using membranes, a bioassay that measures the ability of the molecule to enhance incorporation of tritiated thymidine, in a dose-dependent manner, into the DNA of cells, and the like. Preferably, the variant has at least the same activity as the native molecule.

In addition to the above described FGFs, an agent of use also includes an active fragment of any one of the above-described FGFs. In its simplest form, the active fragment is made by the removal of the N-terminal methionine, using well-known techniques for N-terminal methionine removal, such as a treatment with a methionine aminopeptidase. A second desirable truncation includes an FGF without its leader sequence. Those skilled in the art recognize the leader sequence as the series of hydrophobic residues at the N-terminus of a protein that facilitate its passage through a cell membrane but that are not necessary for activity and that are not found on the mature protein.

Truncations on the FGFs are determined relative to mature FGF-2 having 146 residues. As a general rule, the amino acid sequence of an FGF is aligned with FGF-2 to obtain maximum homology. Portions of the FGF that extend beyond the corresponding N-terminus of the aligned FGF-2 are generally suitable for deletion without adverse effect. Likewise, portions of the FGF that extend beyond the C-terminus of the aligned FGF-2 are also capable of being deleted without adverse effect.

Fragments of FGF that are smaller than those described can also be employed in the present methods. It should be noted that human and murine FGF-2, FGF-4, FGF-8 and a variety of other FGFs, are commercially available.

Suitable biologically active variants can be FGF analogs or derivatives. By “analog” is intended an analog of either FGF or an FGF fragment that includes a native FGF sequence and structure having one or more amino acid substitutions, insertions, or deletions. Analogs having one or more peptoid sequences (peptide mimic sequences) are also included (see, for example, International Publication No. WO 91/04282). By “derivative” is intended any suitable modification of FGF, FGF fragments, or their respective analogs, such as glycosylation, phosphorylation, or other addition of foreign moieties, as long as the FGF activity is retained. Methods for making FGF fragments, analogs and derivatives are available in the art.

In addition to the above-described FGFs, the methods disclosed herein can also employ an active mutant or variant thereof. By the term active mutant, as used in conjunction with an FGF, is meant a mutated form of the naturally occurring FGF. FGF mutant or variants will generally have at least 70%, preferably 80%, more preferably 85%, even more preferably 90% to 95% or more, and for example 98% or more amino acid sequence identity to the amino acid sequence of the reference FGF molecule. A mutant or variant may, for example, differ by as few as 1 to 10 amino acid residues, such as 6-10, as few as 5, as few as 4, 3, 2, or even 1 amino acid residue.

The sequence identity can be determined as described herein. For FGF, one method for determining sequence identify employs the Smith-Waterman homology search algorithm (Meth. Mol. Biol. 70:173-187, 1997) as implemented in MSPRCH program (Oxford Molecular) using an affine gap search with the following search parameters: gap open penalty of 12, and gap extension penalty of 1. In one embodiment, the mutations are “conservative amino acid substitutions” using L-amino acids, wherein one amino acid is replaced by another biologically similar amino acid. Conservative amino acid substitutions are those that preserve the general charge, hydrophobicity, hydrophilicity, and/or steric bulk of the amino acid being substituted.

One skilled in the art, using art known techniques, is able to make one or more point mutations in the DNA encoding any of the FGFs to obtain expression of an FGF polypeptide mutant (or fragment mutant) having an activity for use in methods disclosed herein. To prepare a biologically active mutant of an FGF, one uses standard techniques for site directed mutagenesis, as known in the art and/or as taught in Gilman et al., Gene 8:81, 1979 or Roberts et al., Nature 328:731, 1987, to introduce one or more point mutations into the cDNA that encodes the FGF.

Glial-derived neurotrophic factor (GDNF): A disulfide-bonded homodimeric glycosylated protein of 134 amino acids (18-22 kDa on reducing SDS gels). The amino acid sequences inferred from rat and human cDNAs are 93% identical. The proteins contain seven conserved cysteine residues in the same relative spacing found in all members of the TGF-beta superfamily of proteins. The human GDNF gene has been mapped to chromosome 5p13.1-p13.3. In embryonic midbrain cultures GDNF promotes the survival and morphological differentiation of dopaminergic neurons and increases their high-affinity dopamine uptake. It does not increase total numbers of neurons or astrocytes and does not influence transmitter uptake by serotoninergic neurons. GDNF also has distinct functions outside the nervous system, promoting ureteric branching in kidney development and regulating spermatogenesis. An exemplary amino acid sequence for GDNF can be found as GNEBANK® Accession No. CAG46721 (Jun. 29, 2004), which is incorporated herein by reference.

Suitable biologically active variants can be GDNF analogs or derivatives. By “analog” is intended an analog of either GDNF or an GDNF fragment that includes a native GDNF sequence and structure having one or more amino acid substitutions, insertions, or deletions. Analogs having one or more peptoid sequences (peptide mimic sequences) are also included (see International Patent Publication No. WO 91/04282). By “derivative” is intended any suitable modification of GDNF or a fragment or analog, such as glycosylation, phosphorylation, or other addition of foreign moieties, as long as the GDNF activity is retained. Methods for making GDNF fragments, analogs and derivatives are available in the art. The methods disclosed herein can also employ an active mutant or variant thereof. The term “active GDNF” includes mutated forms of the naturally occurring GDNF. GDNF variants will generally have at least 70%, preferably 80%, more preferably 85%, even more preferably 90% to 95% or more, and for example 98% or more amino acid sequence identity to the amino acid sequence of the reference GDNF molecule. A mutant or variant may, for example, differ by as few as 1 to 10 amino acid residues, such as 6-10, as few as 5, as few as 4, 3, 2, or even 1 amino acid residue.

The sequence identity can be determined as described herein. For GDNF, one method for determining sequence identify employs the Smith-Waterman homology search algorithm (Meth. Mol. Biol. 70:173-187, 1997) as implemented in MSPRCH program (Oxford Molecular) using an affine gap search with the following search parameters: gap open penalty of 12, and gap extension penalty of 1. In one embodiment, the mutations are “conservative amino acid substitutions” using L-amino acids, wherein one amino acid is replaced by another biologically similar amino acid. Conservative amino acid substitutions are those that preserve the general charge, hydrophobicity, hydrophilicity, and/or steric bulk of the amino acid being substituted.

One skilled in the art, using art known techniques, is able to make one or more point mutations in the DNA encoding GDNF to obtain expression of a GDNF polypeptide mutant (or fragment mutant) having an activity for use in methods disclosed herein. Standard techniques for site directed mutagenesis, as known in the art (see, for example, Gilman et al., Gene 8:81, 1979 or Roberts et al., Nature 328:731, 1987) to introduce one or more point mutations into the cDNA that encodes the GDNF.

Growth factor: A substance that promotes cell growth, survival, and/or differentiation. Growth factors include molecules that function as growth stimulators (mitogens), factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors are a fibroblast growth factor (such as FGF-2), epidermal growth factor (EGF), cilliary neurotrophic factor (CNTF), and nerve growth factor (NGF), and actvin-A. In one specific, non-limiting example, a growth factor is insulin. The term “insulin” encompasses naturally occurring insulins and insulin analogs and derivatives. The insulin molecule has been highly conserved in evolution and generally consists of two chains of amino acids linked by disulfide bonds. In humans, insulin is a two-chain insulin molecule (mw 5,800 Daltons), the A-chain is composed of 21 amino acid residues and has glycine at the amino terminus and the B-chain has 30 amino acid residues and phenylalanine at the amino terminus (see U.S. Pat. No. 3,528,960, incorporated herein by reference). Suitable derivatives are known in the art, and include those described in PCT Publication No. WO/12817; U.S. Pat. No. 3,528,960; U.S. Pat. No. 7,229,964; U.S. Pat. No. 7,169,889; U.S. Pat. No. 5,359,030; and U.S. Pat. No. 5,681,811, which are all incorporated herein by reference. Insulin can be isolated from its natural environment or can be synthetic, or genetically engineered (e.g., recombinant) sources. In various embodiments, the insulin is human insulin.

Suitable biologically active variants can be insulin analogs or derivatives. Insulin analogs include a native insulin sequence and structure having one or more amino acid substitutions, insertions, or deletions. Analogs having one or more peptoid sequences (peptide mimic sequences) are also included (see International Patent Publication No. WO 91/04282). By “derivative” is intended any suitable modification of insulin or a fragment or analog, such as glycosylation, phosphorylation, or other addition of foreign moieties, as long as the insulin activity is retained. Methods for making insulin fragments, analogs and derivatives are available in the art (see above). The term “active insulin” includes mutated forms of the naturally occurring insulin, and derivatives of insulin. Insulin variants will generally have at least 70%, preferably 80%, more preferably 85%, even more preferably 90% to 95% or more, and for example 98% or more amino acid sequence identity to the amino acid sequence of the reference insulin molecule. A mutant or variant may, for example, differ by as few as 1 to 10 amino acid residues, such as 6-10, as few as 5, as few as 4, 3, 2, or even 1 amino acid residue.

The sequence identity can be determined as described herein. For insulin, one method for determining sequence identify employs the Smith-Waterman homology search algorithm (Meth. Mol. Biol. 70:173-187, 1997) as implemented in MSPRCH program (Oxford Molecular) using an affine gap search with the following search parameters: gap open penalty of 12, and gap extension penalty of 1. In one embodiment, the mutations are “conservative amino acid substitutions” using L-amino acids, wherein one amino acid is replaced by another biologically similar amino acid. Conservative amino acid substitutions are those that preserve the general charge, hydrophobicity, hydrophilicity, and/or steric bulk of the amino acid being substituted.

One skilled in the art, using art known techniques, is able to make one or more point mutations in the DNA encoding insulin to obtain a polypeptide mutant (or fragment mutant) having an activity for use in methods disclosed herein. Standard techniques for site directed mutagenesis, as known in the art (see, for example, Gilman et al., Gene 8:81, 1979 or Roberts et al., Nature 328:731, 1987) to introduce one or more point mutations into the cDNA that encodes the insulin.

Growth medium or expansion medium: A synthetic set of culture conditions with the nutrients necessary to support the growth (cell proliferation/expansion) of a specific population of cells. In one embodiment, the cells are stem cells, such as ES cells or neuronal stem cells. Growth media generally include a carbon source, a nitrogen source and a buffer to maintain pH. In one embodiment, ES growth medium contains a minimal essential media, such as DMEM, supplemented with various nutrients to enhance ES cell growth. Additionally, the minimal essential media may be supplemented with additives such as horse, calf or fetal bovine serum.

Hairy and Enhancer of Split3 (Hes3): The Hes gene family members are mammalian homologues of the Drosophila hairy and Enhancer of split genes. Hairy and Enhancer of Split function in both segmentation and in the Notch neurogenic pathway during Drosophila embryo development. A conserved role for the Hes genes is in the Notch signaling pathway. During early development of the central nervous system, Hes3 is expressed in the region of the midbrain/hindbrain boundary, and in rhombomeres 2, 4, 6 and 7. Later in development, Hes3 is co-expressed with other neurogenic gene homologues in the developing central nervous system and epithelial cells undergoing mesenchyme induction. An exemplary human Hes3 sequence is set forth as GENBANK® Accession No. NM_{—}001024598 and an exemplary murine Hes3 sequence is set forth s GENBANK® Accession No. NM_{—}008237, both of which are incorporated by reference herein in their entirety.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Hybridization: A process wherein oligonucleotides and their analogs bind by hydrogen bonding, which includes Watson-Crick, Hoogsteen or reversed Hoogsteen hydrogen bonding, between complementary bases. Generally, nucleic acid consists of nitrogenous bases that are either pyrimidines (Cytosine (C), uracil (U), and thymine (T)) or purines (adenine (A) and guanine (G)). These nitrogenous bases form hydrogen bonds consisting of a pyrimidine bonded to a purine, and the bonding of the pyrimidine to the purine is referred to as “base pairing.” More specifically, A will bond to T or U, and G will bond to C. “Complementary” refers to the base pairing that occurs between two distinct nucleic acid sequences or two distinct regions of the same nucleic acid sequence. In one embodiment, nucleic acids that encode growth factors or Notch can be used to produce these proteins. Sequences that hybridize to these nucleic acid molecules, that produce functional proteins, can also be used to produce Notch ligands or growth factors.

“Specifically hybridizable” and “specifically complementary” are terms which indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions in which specific binding is desired, for example under physiological conditions in the case of in vivo assays. Such binding is referred to as “specific hybridization.”

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization.

Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch. The parameters of salt concentration and temperature can be varied to achieve the optimal level of identity between the probe and the target nucleic acid. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference.

For purposes of this disclosure, “stringent conditions” encompass conditions under which hybridization will only occur if there is less than 30% mismatch between the hybridization molecule and the target sequence. “Stringent conditions” may be broken down into particular levels of stringency for more precise definition. Thus, as used herein, “moderate stringency” conditions are those under which molecules with more than 30% sequence mismatch will not hybridize; conditions of “medium stringency” are those under which molecules with more than 20% mismatch will not hybridize, and conditions of “high stringency” are those under which sequences with more than 10% mismatch will not hybridize.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. Similarly, an “isolated” cell has been substantially separated, produced apart from, or purified away from other cells of the organism in which the cell naturally occurs. Isolated cells can be, for example, at least 99%, at least 98%, at least 95%, at least 90%, at least 85%, or at least 80% pure.

Janus Activated Kinase (JAK)/Signal Transducer and Activator of Transcription (STAT): JAKs are cytoplasmic tyrosine kinases that are either constitutively associated with cytokine receptors or recruited to receptors after ligand binding. In either case, stimulation with the ligand results in the catalytic activation of receptor-associated JAKs. This activation results in the phosphorylation of cellular substrates, including the JAK-associated cytokine receptor chains. Some of these phosphorylated tyrosines can serve as coding sites for STAT proteins, which bind to the phosphotyrosines by their SRC-homology 2 (SH2) domains. STAT proteins are also phosphorylated on a conserved tyrosine residue (tyrosine 705 in STAT3), resulting in their dimerization and acquisition of high-affinity DNA-binding activity, which facilitates their action as nuclear transcription factors.

STAT3 is a major cell signaling constituent with roles in both survival and differentiation. However, STAT3 can be phosphorylated on two major residues, Tyrosine (Tyr)705 and Serine (Ser)727. Tyr705 phosphorylation is mediated by JAK2 and Src kinases. Ser727 phosphorylation is mediated by ERK, JNK kinases, TAK1-NLK kinases, and mTOR. Akt and mTOR are also known to mediate survival and growth in many cell types.

The JAK/STAT pathway is one of the most rapid cytoplasmic to nuclear signaling mechanisms. There are a total of four JAK (JAK1-3 and tyrosine kinase 2) and seven STAT proteins (STAT1-4, STAT5A, STAT5b and STAT6). JAKs are relatively large cytoplasmic kinases of about 1,100 amino acids in length, and range in size from about 116 kDa to about 140 kDa. The STAT proteins can dimerize, translocate to the nucleus, and bind DNA. Binding of the STAT proteins to the DNA can result in the activation of transcription (for review see Leonard, Nature Reviews 1: 200-208, 2001).

“STAT inhibitor,” “JAK inhibitor,” and “JAK/STAT inhibitor” are used to refer to any agent capable of down-regulating or otherwise decreasing or suppressing the amount and/or activity of JAK-STAT interactions. JAK inhibitors down-regulate the quantity or activity of JAK molecules. STAT inhibitors down-regulate the quantity or activity of STAT molecules. Inhibition of these cellular components can be achieved by a variety of mechanisms known in the art, including, but not limited to binding directly to JAK (for example, a JAK-inhibitor compound binding complex, or substrate mimetic), binding directly to STAT, or inhibiting the expression of the gene, which encodes the cellular components. JAK/STAT inhibitors are disclosed in U.S. Patent Publication No. 2004/0209799).

Kinase: An enzyme that catalyzes the transfer of a phosphate group from one molecule to another. Kinases play a role in the regulation of cell proliferation, differentiation, metabolism, migration, and survival. A “serine threonine kinase” transfers phosphate groups to a hydroxyl group of serine and/or threonine in a polypeptide.

Receptor protein tyrosine kinases (PTKs) contain a single polypeptide chain with a transmembrane segment. The extracellular end of this segment contains a high affinity ligand-binding domain, while the cytoplasmic end comprises the catalytic core and the regulatory sequences. The cytosolic end also contains tyrosine residues, which become substrates or targets for the tyrosine kinase portion of the receptor. PTK remains inactive until a ligand binds to the receptor, which leads to the dimerization of two ligand-bound receptors (exception: insulin receptor). Once activated, receptors are able to autophosphorylate tyrosine residues outside the catalytic domain. This stabilizes the active receptor conformation and creates phosphotyrosine-docking sites for proteins that transduce signals within the cell. The cytosolic portion of the phosphorylated receptor recruits a number of cytosolic adapter proteins via interactions between phosphorylated tyrosine residues on the receptor and the SH2 domain on the adapter molecule. Different proteins have different SH2 domains that recognize specific phosphotyrosine residues. An SH2-containing protein, Grb2, acts as a common adapter protein in a majority of growth factor related signaling events.

Non-receptor tyrosine kinases include members of the Src, Tec, JAK, Fes, Abl, FAK, Csk, and Syk families. They are located in the cytoplasm as well as in the nucleus. They exhibit distinct kinase regulation, substrate phosphorylation, and function. In most cases, their activation also begins with the phosphorylation of a tyrosine residue present in an activation loop.

One example of a kinase is a JAK (see above). Another example of a kinase is a “phosphatidyl inositol 3-kinase,” an enzyme that phosphorylates inositol lipids at the D-3 position of the inositol ring to generate the 3-phosphoinositides, phosphatidylinositol 3-phosphate [PtdIns(3)P], phosphatidylinositol 3,4-bisphosphate [PtdIns(3,4)P₂] and phosphatidylinositol 3,4,5-trisphosphate [PtdIns(3,4,5)P₃]. GENBANK® Accession No. AAB53966 (May 9, 1997) sets forth an exemplary amino acid sequence of the catalytic subunit of human phosphatidyl inositol 3-kinase. A “preferential” inhibition of a kinase refers to decreasing activity of one kinase, such as MAP kinase (see below), more than inhibiting the activity of a second kinase, such as JAK.

Mitogen-activated protein kinases (MAP Kinases): A group of protein serine/threonine kinases that are activated in response to a variety of extracellular stimuli and mediate signal transduction from the cell surface to the nucleus. In combination with several other signaling pathways, they can differentially alter phosphorylation status of the transcription factors. A controlled regulation of these cascades is involved in cell proliferation and differentiation.

The p38 kinase (“p38”) is the most well-characterized member of the MAP kinase family. It is activated in response to inflammatory cytokines, endotoxins, and osmotic stress. It shares about 50% homology with the ERKs. However, downstream activation of p38 occurs following its phosphorylation (at the TGY motif) by MKK3, a dual specificity kinase. Following its activation, p38 translocates to the nucleus and phosphorylates ATF-2.

Neurological disorder: A disorder in the nervous system, including the central nervous system (CNS) and peripheral nervous system (PNS). Examples of neurological disorders include Parkinson's disease, Huntington's disease, Alzheimer's disease, severe seizure disorders including epilepsy, familial dysautonomia as well as injury or trauma to the nervous system, such as neurotoxic injury or disorders of mood and behavior such as addiction, schizophrenia and amyotrophic lateral sclerosis. Neuronal disorders also include Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia.

Neurodegenerative disorder: An abnormality in the nervous system of a subject, such as a mammal, in which neuronal integrity is threatened. Without being bound by theory, neuronal integrity can be threatened when neuronal cells display decreased survival or when the neurons can no longer propagate a signal. Specific, non-limiting examples of a neurodegenerative disorder are Alzheimer's disease, Pantothenate kinase associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis.

Alzheimer's disease manifests itself as pre-senile dementia. The disease is characterized by confusion, memory failure, disorientation, restlessness, speech disturbances, and hallucination in mammals (Medical, Nursing, and Allied Health Dictionary, 4th Ed., 1994, Editors: Anderson, Anderson, Glanze, St. Louis, Mosby). Alzheimer's disease is characterized by a progressive loss of neurons, formation of fibrillary tangles within neurons and numerous plaques in affected brain regions. It is believed that the key pathogenic event in Alzheimer's disease is the excessive formation and/or accumulation of fibrillar β-amyloid peptides, which are also called αβ.

Parkinson's disease is a slowly progressive, degenerative, neurologic disorder characterized by resting tremor, loss of postural reflexes, and muscle rigidity and weakness (Medical, Nursing, and Allied Health Dictionary, 4th Ed., 1994, Editors: Anderson, Anderson, Glanze, St. Louis, Mosby).

Amyotrophic lateral sclerosis is a degenerative disease of the motor neurons characterized by weakness and atrophy of the muscles of the hands, forearms and legs, spreading to involve most of the body and face (Medical, Nursing, and Allied Health Dictionary, 4th Ed., 1994, Editors: Anderson, Anderson, Glanze, St. Louis, Mosby).

Pantothenate kinase associated neurodegeneration (PKAN, also known as Hallervorden-Spatz syndrome) is an autosomal recessive neurodegenerative disorder associated with brain iron accumulation. Clinical features include extrapyramidal dysfunction, onset in childhood, and a relentlessly progressive course (Dooling et al., Arch. Neurol. 30:70-83, 1974). PKAN is a clinically heterogeneous group of disorders that includes classical disease with onset in the first two decades, dystonia, high globus pallidus iron with a characteristic radiographic appearance (Angelini et al., J. Neurol. 239:417-425, 1992), and often either pigmentary retinopathy or optic atrophy (Dooling et al., Arch. Neurol. 30:70-83, 1974; Swaiman et al., Arch. Neurol 48:1285-1293, 1991).

A “neurodegenerative related disorder” is a disorder such as speech disorders that are associated with a neurodegenerative disorder. Specific non-limiting examples of a neurodegenerative related disorders include, but are not limited to, palilalia, tachylalia, echolalia, gait disturbance, preservative movements, bradykinesia, spasticity, rigidity, retinopathy, optic atrophy, dysarthria, and dementia.

Nestin: A protein whose expression distinguishes neural multi-potential stem cells and brain tumor cells from the more differentiated neural cell types (such as neuronal, glial and muscle cells) of the mammalian brain. Nestin is an intermediate filament. The similarity between the nestin gene and the genes of the other five classes of intermediate filaments ranges from 16% to 29% at the amino acid level in a 307 amino acid long region starting close to the N-terminus of the nestin gene, corresponding to the conserved alpha-helical rod or “core” domain of the intermediate filaments. This region of the predicted nestin amino acid sequence also contains a repeated hydrophobic heptad motif characteristic of intermediate filaments. Amino acid sequences of nestin are disclosed, for example, in U.S. Pat. No. 5,338,839, which is incorporated herein by reference.

Notch: An integral membrane protein of 2703 amino acids that was first identified in Drosophilia. Notch is the Drosophila homologue of the human epidermal growth factor (EGF) ceptor. Mammals have more than one Notch gene homolog. The Notch-1 gene is located human chromosome 9q34; the structure of Notch-1 is similar to Notch-2 (found on human chromosome 1p13-p11). Notch-3 (found on human chromosome 19p13.2-p13.1) lacks some of the domains found in the other family members and encodes a considerably shorter intracellular domain.

The intracellular domain of Notch has a length of approximately 1000 amino acids and is composed of a number of different sequence domains. The extracellular domain of wild-type Notch contains 36 EGF-like repeats that differ slightly in sequence. Some of these repeats are involved in the dimerisation and multimerisation of the Notch protein. Other repeats function as receptor domains for proteins involved in the differentiation of cells into neural and epidermal precursors.

Exemplary Notch amino acid sequences are as follows:

| Notch Protein | GENBANK ® Accession No.¹ |
|---|---|
| Human Notch 1 | NM_017617 (Sep. 3, 2006) |
| Human Notch 2 | NM_024408 (Aug. 28, 2006) |
| Human Notch 3 | NM_000435 (Sep. 3, 2006) |
| Human Notch 4 | NM_004557 (Aug. 28, 2006) |
| Mouse Notch 1 | NM_008714 (Aug. 20, 2006) |
| Mouse Notch 2 | NM_010928 (Aug. 6, 2006) |
| Mouse Notch 3 | NM_008716 (Aug. 6, 2006) |
| Mouse Notch 4 | NM_010929 (Aug. 6, 2006) |

¹All GENBANK ® data is incorporated by reference herein. Dates expressed as month-day-year.

Two of the 36 EGF-repeats of the extracellular domain of Notch interact with another protein, called Delta and with other proteins, Serrate, and Lag-2, These proteins are collectively referred to also Notch ligands or DSL ligands. Jagged (also called Serrate-1) is also a Notch ligand. (see Artavanis-Tsakonas et al., Annual Review of Cell Biology 7: 427-452, 1991; U.S. Pat. No. 6,083,904, U.S. Pat. No. 6,149,902, and U.S. Pat. No. 5,780,3000, which are herein incorporated by reference. Delta proteins and nucleic acids are disclosed in U.S. Pat. No. 6,783,956, which is incorporated herein by reference.

Notch ligands such as Delta are of use in the methods disclosed herein. Suitable biologically active variants can be Notch ligand analogs or derivatives. By “analog” is intended an analog of either Notch ligand or a Notch ligand fragment that includes a native Notch ligand sequence and structure having one or more amino acid substitutions, insertions, or deletions. Analogs having one or more peptoid sequences (peptide mimic sequences) are also included (see International Patent Publication No. WO 91/04282). By “derivative” is intended any suitable modification of Notch ligand or a fragment or analog, such as glycosylation, phosphorylation, or other addition of foreign moieties, as long as the Notch ligand activity is retained. Methods for making Notch ligand fragments, analogs and derivatives are available in the art. In addition to the above-described Notch ligand, the methods disclosed herein can also employ an active mutant or variant thereof. The term “active Notch ligand” includes mutated forms of the naturally occurring Notch ligand. Notch ligand variants will generally have at least 70%, preferably 80%, more preferably 85%, even more preferably 90% to 95% or more, and for example 98% or more amino acid sequence identity to the amino acid sequence of the reference Notch ligand molecule. A mutant or variant may, for example, differ by as few as 1 to 10 amino acid residues, such as 6-10, as few as 5, as few as 4, 3, 2, or even 1 amino acid residue.

The sequence identity can be determined as described herein. For a Notch ligand, one method for determining sequence identify employs the Smith-Waterman homology search algorithm (Meth. Mol. Biol. 70:173-187, 1997) as implemented in MSPRCH program (Oxford Molecular) using an affine gap search with the following search parameters: gap open penalty of 12, and gap extension penalty of 1. In one embodiment, the mutations are “conservative amino acid substitutions” using L-amino acids, wherein one amino acid is replaced by another biologically similar amino acid. Conservative amino acid substitutions are those that preserve the general charge, hydrophobicity, hydrophilicity, and/or steric bulk of the amino acid being substituted.

One skilled in the art, using art known techniques, is able to make one or more point mutations in the DNA encoding Ang-2 to obtain expression of a Notch ligand polypeptide mutant (or fragment mutant) having an activity for use in methods disclosed herein. Standard techniques for site directed mutagenesis, as known in the art (see, for example, Gilman et al., Gene 8:81, 1979 or Roberts et al., Nature 328:731, 1987) to introduce one or more point mutations into the cDNA that encodes the Notch ligand.

Nucleotide: A monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Peripheral Nervous System (PNS): The part of an animal's nervous system other than the Central Nervous System. Generally, the PNS is located in the peripheral parts of the body and includes cranial nerves, spinal nerves and their branches, and the autonomic nervous system.

Polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide, such as a nucleostemin. Biologically functional fragments, for example, can vary in size from a polypeptide fragment as small as an epitope capable of binding an antibody molecule to a large polypeptide capable of participating in the characteristic induction or programming of phenotypic changes within a cell, including affecting cell proliferation or differentiation. An “epitope” is a region of a polypeptide capable of binding an immunoglobulin generated in response to contact with an antigen. Thus, smaller peptides containing the biological activity of insulin, or conservative variants of the insulin, are thus included as being of use. The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions (or “conservative variants”) replace one amino acid with another amino acid that is similar in size, hydrophobicity, etc. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Variations in the cDNA sequence that result in amino acid changes, whether conservative or not, should be minimized in order to preserve the functional and immunologic identity of the encoded protein. Thus, in several non-limiting examples, a nucleostemin polypeptide includes at most two, at most five, at most ten, at most twenty, or at most fifty conservative substitutions. The immunologic identity of the protein may be assessed by determining whether it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may, for example, be 80%, 90% or even 95% or 98% identical to the native amino acid sequence.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Pharmaceutical agent: A chemical compound, small molecule, or other composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded by a recombinant nucleic acid molecule.

Senescence: The inability of a cell to divide further. A senescent cell is still viable, but does not divide.

Sequence identity: The similarity between amino acid sequences, such as growth factor or Notch ligand amino acid sequences, is expressed in terms of the percentage of conservation between the sequences, otherwise referred to as sequence similarity. Sequence identity is frequently measured in terms of percentage identity (or similarity or homology); the higher the percentage, the more similar the two sequences are. Homologues or variants of a growth factor or a Notch ligand will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet., 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the Internet, for use in connection with the sequence analysis programs blastp, blastn, blastx, tblastn and tblastx. A description of how to determine sequence identity using this program is available on the NCBI website on the Internet. Other specific, non-limiting examples of sequence alignment programs specifically designed to identify conserved regions of genomic DNA of greater than or equal to 100 nucleotides are PIPMaker (Schwartz et al., Genome Research 10: 577-586, 2000) and DOTTER (Erik et al., Gene 167: GC1-10, 1995).

Homologues and variants of a nucleic acid sequence are typically characterized by possession of at least 75%, for example at least 80%, 90%, 95%, 98%, or 99%, sequence identity counted over the full length alignment with the originating sequence using the NCBI Blast 2.0, set to default parameters. Methods for determining sequence identity over such short windows are available at the NCBI website on the Internet. One of skill in the art will appreciate that these sequence identity ranges are provided for guidance only; it is entirely possible that strongly significant homologues could be obtained that fall outside of the ranges provided.

Stem cell: A cell that can generate a fully differentiated functional cell of a more than one given cell type. The role of stem cells in vivo is to replace cells that are destroyed during the normal life of an animal. Generally, stem cells can divide without limit and are totipotent or pluripotent. After division, the stem cell may remain as a stem cell, become a precursor cell, or proceed to terminal differentiation. A nervous system (NS) stem cell is, for example, a cell of the central nervous system that can self-renew and can generate astrocytes, neurons and oligodendrocytes.

A “somatic precursor cell” is a cell that can generate a fully differentiated functional cell of at least one given cell type from the body of an animal, such as a human. A neuronal precursor cell can generate of fully differentiated neuronal cell, such as, but not limited to, and adrenergic or a cholinergic neuron. A glial precursor cell can generate fully differentiated glial cells, such as but not limited to astrocytes, microglia and oligodendroglia. Generally, precursor cells can divide and are pluripotent. After division, a precursor cell can remain a precursor cell, or may proceed to terminal differentiation. A neuronal precursor cell can give rise to one or more types of neurons, such as dopaminergic, adrenergic, or serotinergic cells, but is more limited in its ability to differentiate than a stem cell. In one example, a neuronal stem cell gives rise to all of the types of neuronal cells (such as dopaminergic, adrenergic, and serotinergic neurons) but does not give rise to other cells, such as glial cells.

Sonic hedgehog (SHH): Sonic hedgehog (SHH) is one of three mammalian homologs of the Drosophila hedgehog signaling molecule and is expressed at high levels in the notochord and floor plate of developing embryos. SHH is known to play a key role in neuronal tube patterning (Echerlard et al., Cell 75:1417-30, 1993), the development of limbs, somites, lungs and skin. Moreover, overexpression of SHH has been found in basal cell carcinoma. Exemplary amino acid sequences of SHH is set forth in U.S. Pat. No. 6,277,820.

Subject: Any mammal, such as humans, non-human primates, pigs, sheep, cows, rodents and the like, which is to be the recipient of the particular treatment. In one embodiment, a subject is a human subject or a murine subject.

Survival (of a Cell): The length of time a given cell is alive. An increase in survival following treatment indicates that the cell lives for a longer length of time as compared to a control, such as the cell in the absence of treatment.

Synapse: Highly specialized intercellular junctions between neurons and between neurons and effector cells across which a nerve impulse is conducted (synaptically active). Generally, the nerve impulse is conducted by the release from one neuron (presynaptic neuron) of a chemical transmitter (such as dopamine or serotonin) which diffuses across the narrow intercellular space to the other neuron or effector cell (post-synaptic neuron). Generally neurotransmitters mediate their effects by interacting with specific receptors incorporated in the post-synaptic cell. “Synaptically active” refers to cells (e.g., differentiated neurons) which receive and transmit action potentials characteristic of mature neurons.

Tie-2: A receptor tyrosine kinase (RTK) originally was described as the second member of an orphan RTK subfamily expressed predominantly in the embryonic endothelium. Wild-type Tie-1 and Tie-2 both have an N-terminal ligand-binding domain, a single transmembrane domain, and an intracellular tyrosine kinase domain. The domain structure of Tie2 is highly conserved from zebra fish to human, with the greatest amino acid homology occurring in the kinase domain. Ang1 binding stimulates autophosphorylation of the kinase domain of Tie2. However, Ang2 does not stimulate Tie2 autophosphorylation but instead blocked Ang1-mediated Tie2 activation and endothelial migration. This suggests that in some circumstances in embryonic cells Ang2 is a naturally occurring inhibitor of Tie2 activation. However, in other circumstances, Ang2 can stimulate Tie2, suggesting that the action of Ang2 as a Tie2 agonist or antagonist is context dependent in embryonic cells. Tie2 pathway has important functions in adult tissues, in both quiescent vasculature and during angiogenesis (for review, see Peters et al., Recent progress in Hormone Research 59: 51-71, 2004). An exemplary amino acid sequence for human Tie-2 is set forth in GENBANK® Accession No. Q02763 (Jun. 10, 2008, herein incorporated by reference).

Therapeutic agent: Used in a generic sense, it includes treating agents, prophylactic agents, and replacement agents.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an artificial islet produced by the methods described herein.

Genetic modification of the target cell is one indicia of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Methods for Increasing the Survival and/or Proliferation of Stem Cells and Progenitor Cells

Methods are disclosed herein for increasing the number of stem cells or precursor cells. The number of stem cells can be increased by increasing survival and/or proliferation of the cells. The methods include contacting the cells with an effective amount of a Notch ligand, an effective amount of a growth factor, and an effective amount of angiopoietin-2 (Ang-2). In several embodiments, the methods include contacting the cells with an effective amount of a Jak inhibitor. In several examples, the methods result in increased survival and/or increased proliferation of stem cells and/or precursor cells. The cells can be pluripotent or totipotent, and can be in vivo or in vitro. In several non-limiting examples, the cells are neuronal stem cells or neuronal precursor cells.

### Cells

In one example, the cells are stem cells, such as embryonic stem cells. For example, murine, primate or human cells can be utilized. In several examples, the cells are embryonic stem (ES) cells, which can proliferate indefinitely in an undifferentiated state. Furthermore, ES cells are totipotent cells, meaning that they can generate all of the cells present in the body (bone, muscle, brain cells, etc.). ES cells have been isolated from the inner cell mass (ICM) of the developing murine blastocyst (Evans et al., Nature 292:154-156, 1981; Martin et al., Proc. Natl. Acad. Sci. 78:7634-7636, 1981; Robertson et al., Nature 323:445-448, 1986). Additionally, human cells with ES properties have been isolated from the inner blastocyst cell mass (Thomson et al., Science 282:1145-1147, 1998) and developing germ cells (Shamblott et al., Proc. Natl. Acad. Sci. USA 95:13726-13731, 1998), and human and non-human primate embryonic stem cells have been produced (see U.S. Pat. No. 6,200,806, which is incorporated by reference herein).

As disclosed in U.S. Pat. No. 6,200,806, ES cells can be produced from human and non-human primates. In one embodiment, primate ES cells are isolated “ES medium” that express SSEA-3; SSEA-4, TRA-1-60, and TRA-1-81 (see U.S. Pat. No. 6,200,806). ES medium consists of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). Generally, primate ES cells are isolated on a confluent layer of murine embryonic fibroblast in the presence of ES cell medium. In one example, embryonic fibroblasts are obtained from 12 day old fetuses from out bred mice (such as CF1, available from SASCO), but other strains may be used as an alternative. Tissue culture dishes treated with 0.1% gelatin (type I; Sigma) can be utilized. Distinguishing features of ES cells, as compared to the committed “multipotential” stem cells present in adults, include the capacity of ES cells to maintain an undifferentiated state indefinitely in culture, and the potential that ES cells have to develop into every different cell types. Unlike mouse ES cells, human ES (hES) cells do not express the stage-specific embryonic antigen SSEA-1, but express SSEA-4, which is another glycolipid cell surface antigen recognized by a specific monoclonal antibody (see, for example, Amit et al., Devel. Biol. 227:271-278, 2000).

For rhesus monkey embryos, adult female rhesus monkeys (greater than four years old) demonstrating normal ovarian cycles are observed daily for evidence of menstrual bleeding (day 1 of cycle=the day of onset of menses). Blood samples are drawn daily during the follicular phase starting from day 8 of the menstrual cycle, and serum concentrations of luteinizing hormone are determined by radioimmunoassay. The female is paired with a male rhesus monkey of proven fertility from day 9 of the menstrual cycle until 48 hours after the luteinizing hormone surge; ovulation is taken as the day following the luteinizing hormone surge. Expanded blastocysts are collected by non-surgical uterine flushing at six days after ovulation. This procedure generally results in the recovery of an average 0.4 to 0.6 viable embryos per rhesus monkey per month (Seshagiri et al., Am J Primatol. 29:81-91, 1993).

For marmoset embryos, adult female marmosets (greater than two years of age) demonstrating regular ovarian cycles are maintained in family groups, with a fertile male and up to five progeny. Ovarian cycles are controlled by intramuscular injection of 0.75 g of the prostaglandin PGF2a analog cloprostenol (Estrumate, Mobay Corp, Shawnee, Kans.) during the middle to late luteal phase. Blood samples are drawn on day 0 (immediately before cloprostenol injection), and on days 3, 7, 9, 11, and 13. Plasma progesterone concentrations are determined by ELISA. The day of ovulation is taken as the day preceding a plasma progesterone concentration of 10 ng/ml or more. At eight days after ovulation, expanded blastocysts are recovered by a non-surgical uterine flush procedure (Thomson et al., J Med. Primatol. 23:333-336, 1994). This procedure results in the average production of 1.0 viable embryos per marmoset per month.

The zona pellucida is removed from blastocysts, such as by brief exposure to pronase (Sigma). For immunosurgery, blastocysts are exposed to a 1:50 dilution of rabbit anti-marmoset spleen cell antiserum (for marmoset blastocysts) or a 1:50 dilution of rabbit anti-rhesus monkey (for rhesus monkey blastocysts) in DMEM for 30 minutes, then washed for 5 minutes three times in DMEM, then exposed to a 1:5 dilution of Guinea pig complement (Gibco) for 3 minutes. After two further washes in DMEM, lysed trophectoderm cells are removed from the intact inner cell mass (ICM) by gentle pipetting, and the ICM plated on mouse inactivated (3000 rads gamma irradiation) embryonic fibroblasts.

After 7-21 days, ICM-derived masses are removed from endoderm outgrowths with a micropipette with direct observation under a stereo microscope, exposed to 0.05% Trypsin-EDTA (Gibco) supplemented with 1% chicken serum for 3-5 minutes and gently dissociated by gentle pipetting through a flame polished micropipette.

Dissociated cells are re-plated on embryonic feeder layers in fresh ES medium, and observed for colony formation. Colonies demonstrating ES-like morphology are individually selected, and split again as described above. The ES-like morphology is defined as compact colonies having a high nucleus to cytoplasm ratio and prominent nucleoli. Resulting ES cells are then routinely split by brief trypsinization or exposure to Dulbecco's Phosphate Buffered Saline (PBS, without calcium or magnesium and with 2 mM EDTA) every 1-2 weeks as the cultures become dense. Early passage cells are also frozen and stored in liquid nitrogen.

Cell lines may be karyotyped with a standard G-banding technique (such as by the Cytogenetics Laboratory of the University of Wisconsin State Hygiene Laboratory, which provides routine karyotyping services) and compared to published karyotypes for the primate species.

Isolation of ES cell lines from other primate species would follow a similar procedure, except that the rate of development to blastocyst can vary by a few days between species, and the rate of development of the cultured ICMs will vary between species. For example, six days after ovulation, rhesus monkey embryos are at the expanded blastocyst stage, whereas marmoset embryos do not reach the same stage until 7-8 days after ovulation. The rhesus ES cell lines can be obtained by splitting the ICM-derived cells for the first time at 7-16 days after immunosurgery; whereas the marmoset ES cells were derived with the initial split at 7-10 days after immunosurgery. Because other primates also vary in their developmental rate, the timing of embryo collection, and the timing of the initial ICM split, varies between primate species, but the same techniques and culture conditions will allow ES cell isolation (see U.S. Pat. No. 6,200,806, which is incorporated herein by reference for a complete discussion of primate ES cells and their production).

Human ES cell lines exist and can be used in the methods disclosed herein. Human ES cells can also be derived from preimplantation embryos from in vitro fertilized (IVF) embryos. Experiments on unused human IVF-produced embryos are allowed in many countries, such as Singapore and the United Kingdom, if the embryos are less than 14 days old. Only high quality embryos are suitable for ES isolation. Present defined culture conditions for culturing the one cell human embryo to the expanded blastocyst have been described (see Bongso et al., Hum Reprod. 4:706-713, 1989). Co-culturing of human embryos with human oviductal cells results in the production of high blastocyst quality. IVF-derived expanded human blastocysts grown in cellular co-culture, or in improved defined medium, allows isolation of human ES cells with the same procedures described above for non-human primates (see U.S. Pat. No. 6,200,806).

Somatic precursor cells can also be utilized with the methods disclosed herein. The somatic precursor cells can be isolated from a variety of sources using methods known to one skilled in the art. The somatic precursor cells can be of ectodermal, mesodermal or endodermal origin. Any somatic precursor cells which can be obtained and maintained in vitro can potentially be used in accordance with the present methods. Such cells include cells of epithelial tissues such as the skin and the lining of the gut, embryonic heart muscle cells, and neural precursor cells (Stemple and Anderson, 1992, Cell 71:973-985). Such cells also include pancreatic stem cells, cord blood stem cells, peripheral blood stem cells, and stem cells derived from adipose tissues.

In on non-limiting example, the cells are neuronal stem cells. In other non-limited examples, the cells are neuronal precursor cells and/or glial precursor cells. Undifferentiated neural stem cells differentiate into neuroblasts and glioblasts which give rise to neurons and glial cells. During development, cells that are derived from the neural tube give rise to neurons and glia of the central nervous system (CNS). Certain factors present during development, such as nerve growth factor (NGF), promote the growth of neural cells. Methods of isolating and culturing neural stem cells and neuronal/glial progenitor cells are well known to those of skill in the art (Hazel and Muller, 1997; U.S. Pat. No. 5,750,376, which are both incorporated herein by reference). Methods for isolating and culturing neuronal precursor cells are disclosed, for example, in U.S. Pat. No. 6,610,540, which is incorporated herein by reference. Methods for the isolating and culturing these cells are also disclosed, without limitation, in the examples section. Thus, methods are disclosed herein for increasing the number of neuronal stem cells, neuronal precursor cells and/or glial precursor cells.

In several examples, methods are disclosed for increasing the number of mesenchymal progenitor cells. Mesenchymal progenitors give rise to a very large number of distinct tissues (Caplan, J. Orth. Res. 641-650, 1991). Mesenchymal cells capable of differentiating into bone and cartilage have also been isolated from marrow (Caplan, J. Orth. Res. 641-650, 1991). U.S. Pat. No. 5,226,914 describes an exemplary method for isolating mesenchymal stem cells from bone marrow. In other examples, the somatic precursor cells are epithelial progenitor cells or keratinocytes can be obtained from tissues such as the skin and the lining of the gut by known procedures (Rheinwald, Meth. Cell Bio. 21A:229, 1980). In stratified epithelial tissue such as the skin, renewal occurs by mitosis of precursor cells within the germinal layer, the layer closest to the basal lamina. Precursor cells within the lining of the gut provide for a rapid renewal rate of this tissue. The cells can also be liver stem cells (see PCT Publication No. WO 94/08598) or kidney stem cells (see Karp et al., Dev. Biol. 91:5286-5290, 1994). The cells can also be inner ear stem cells (see Li et al., TRENDS Mol. Med. 10: 309, 2004).

### Agents of Use in the Methods

The methods disclosed herein include contacting cells of interest with a Notch agonist. Agonists of the Notch pathway are able to activate the Notch pathway at the level of protein-protein interaction or protein-DNA interaction. Agonists of Notch include but are not limited to proteins including portions of toporythmic proteins such asF3/Contactin or Delta or Serrate or Jagged (Lindsell et al., Cell 80:909-917, 1995) that mediate binding to Notch, and nucleic acids encoding the foregoing (which can be administered to express their encoded products in vivo). Thus, agonists of the Notch pathway include, but are not limited to, Notch ligands.

Jagged (also called Serrate-1) is also a Notch ligand (see Artavanis-Tsakonas et al., Annual Review of Cell Biology 7: 427-452, 1991; U.S. Pat. No. 6,083,904, U.S. Pat. No. 6,149,902, and U.S. Pat. No. 5,780,3000, which are herein incorporated by reference). Delta is another Notch ligand. Delta proteins and nucleic acids are disclosed in U.S. Pat. No. 6,783,956, which is incorporated herein by reference.

Exemplary Notch Ligands are shown in the following table:

| Notch Ligand | GENBANK ® Accession No.¹ |
|---|---|
| Human Delta-1 | NM_005618 (Aug. 20, 2006) |
| Human Delta-3 | NM_016941 (Aug. 13, 2006) |
| Human Delta-4 | NM_019074 (Aug. 20, 2006) |
| Human Jagged-1 | NM_000214 (Aug. 20, 2006) |
| Human Jagged-2 | NM_002226 (Aug. 20, 2006) |
| Human DNER | NM_139072 (Aug. 20, 2006) |
| Human F3/contactin | NM_001843 (Aug. 20, 2006) |
| Mouse Delta-1 | NM_007865 (Aug. 27, 2006) |
| Mouse Delta-3 | NM_007866 (Jul. 16, 2006) |
| Mouse Delta-4 | NM_019454 (Jul. 16, 2006) |
| Mouse Jagged-1 | NM_013822 (Aug. 20, 2006) |
| Mouse Jagged-2 | NM_010588 (Jul. 16, 2006) |
| Mouse DNER | NM_152915 (May 14, 2006) |
| Mouse F3/contactin | NM_007727 (Feb. 12, 2006) |

¹All GENBANK ® data is incorporated by refernece herein. Dates expressed as month-day-year.

Conservative variants of these amino acid sequences, such as at most 1, at most 2, at most 3, at most 5 or at most ten conservative amino acid substitutions in the amino acid sequences set forth in the above list are also included, wherein the variants bind Notch and induce cell signalling through Notch, can also be used in the methods disclosed herein. Fragments of these amino acid sequences that bind Notch and induce biological activity through the Notch receptor are also of use in the methods disclosed herein.

In one embodiment, the Notch agonist is a functionally active fragment of a protein, such as a fragment of a Notch ligand that mediates binding to Notch. In another embodiment, the agonist is a full-length protein or portion thereof (such as human Delta). In an additional embodiment, the Notch antagonist is a chimeric protein including a functional fragment of a Notch ligand and a heterologous polypeptide. Nucleic acids encoding these Notch agonists are also of use.

In one example, the Notch agonist is a fusion protein including the extra-cellular domain of Delta and an immunoglobulin constant domain. In yet another embodiment the agonist is Deltex or Suppressor of Hairless. In another embodiment, a recombinant Notch agonist is a chimeric Notch protein which comprises the intracellular domain of Notch and the extracellular domain of another ligand-binding surface receptor. For example, a chimeric Notch protein comprising the EGF receptor extracellular domain and the Notch intracellular domain has been described. Exemplary agonists and ligands are described in detail in U.S. Pat. No. 5,780,300, U.S. Pat. No. 6,703,221, and Murata-Oh et al., Int. J. Molec. Med. 13: 419-423, 2004, which are all incorporated by reference in their entirety. The Notch ligand, fragment thereof, or chimeric Notch protein can include a human or a mouse Notch ligand or fragment thereof. In a further example, a nucleic acid encoding Deltex or Suppressor of Hairless is utilized in the method disclosed herein. It should be noted that any of the Notch ligands described above are of use in any of the methods disclosed herein.

The methods disclosed herein also include contacting the cells with an effective amount of a growth factor. In one embodiment the growth factor is insulin. The amino acid sequence of insulin is well known in the art (see, for example, GENBANK® Accession No. AAA59172, Feb. 12, 2001; GENBANK® Accession No. AAN39451, Sep. 4, 2003, and GENBANK® Accession No. AAH05255, Jun. 23, 2006, which are incorporated herein by reference), and there are several commercial sources of insulin, including human insulin.

Suitable insulin derivatives of use in the methods disclosed herein are known in the art, and include those described in PCT Publication No. WO 96/29998; U.S. Pat. No. 3,528,960; U.S. Pat. No. 7,229,964; U.S. Pat. No. 7,169,889; U.S. Pat. No. 5,359,030; and U.S. Pat. No. 5,681,811, which are all incorporated herein by reference. Insulin can be isolated from its natural environment or can be synthetic, or genetically engineered (e.g., recombinant) sources. In various embodiments, the insulin is human insulin.

Suitable biologically active variants can be insulin analogs or derivatives. Insulin analogs include a native insulin sequence and structure having one or more amino acid substitutions, insertions, or deletions. Analogs having one or more peptoid sequences (peptide mimic sequences) are also included (see International Patent Publication No. WO 91/04282). By “derivative” is intended any suitable modification of insulin or a fragment or analog, such as glycosylation, phosphorylation, or other addition of foreign moieties, as long as the insulin activity is retained. Methods for making insulin fragments, analogs and derivatives are available in the art (see above). It should be noted that insulin can be in the form of proinsulin, which is processed into insulin in a mammalian subject.

In other embodiments, the growth factor is a fibroblast growth factor, such as, but not limited to, FGF-2 or FGF-4, or a fragment or variant thereof. In a further embodiment, the growth factor is GDNF, or a fragment or variant thereof. An exemplary amino acid sequence for GDNF can be found as GNEBANK® Accession No. CAG46721 (Jun. 29, 2004), which is incorporated herein by reference. GDNF and variants of GDNF are further described in U.S. Pat. No. 7,226,758, which is incorporated herein by reference. Combinations of growth factors can also be used, such as a combination of insulin and GDNF. Thus, in specific non-limiting examples, a therapeutically effective amount of Delta and insulin, Jagged and insulin, Delta and GDNF, Jagged and GDNF, Delta and insulin and GDNF, Jagged and insulin and GDNF, are administered to a subject, along with a therapeutically effective amount of Ang-2.

The methods disclosed herein include contacting the cells with an effective amount of angiopoietin-2, or a biologically active fragment or variant thereof (see, for example, PCT Publication No. WO 98/05779, incorporated herein by reference). Exemplary amino acid sequences for human Ang-2 are shown in GENBANK® Accession No. NP_{—}001138, Jul. 30, 2007, and GENBANK® Accession No. BAA95590, May 10, 2000, which are incorporated herein by reference. The sequences of several mammalian Ang-2 proteins are known, including, but no limited to the rat and mouse sequences (see GENBANK® Accession Nos. 035462, Jul. 10, 2007 and NP_{—}031452, Jul. 30, 2007, which are incorporated herein by reference).

In several embodiments, the cells also are contacted with an effective amount of a Janus kinase (JAK) inhibitor. Inhibitors of JAK are well known in the art, see for example, U.S. Pat. No. 6,452,005. In addition, bis monocyclic, bicyclic or heterocyclic aryl compounds (PCT Publication No. WO 92/20642), vinylene-azaindole derivatives (PCT Publication No. WO 94/14808) and 1-cycloproppyl-4-pyridyl-quinolones (U.S. Pat. No. 5,330,992) have been described the use of these agents as tyrosine kinase inhibitors. Styryl compounds (U.S. Pat. No. 5,217,999), styryl-substituted pyridyl compounds (U.S. Pat. No. 5,302,606), certain quinazoline derivatives (published EP Application No. 0 566 266 A1), seleoindoles and selenides (PCT Publication No. WO 94/03427), tricyclic polyhydroxylic compounds (PCT Publication No. WO 92/21660) have also been disclosed to be tyrosine kinase inhibitors. An exemplary JAK inhibitor is 2-(1,1-Dimethylethyl)-9-fluoro-3,6-dihydro-7H-benz[h]-imidaz[4,5-f]isoquinolin-7-one. A JAK inhibitor and a p38 inhibitor can be used in combination in the methods disclosed herein.

### Methods for Increasing Cell Number: In Vivo and In Vitro

Methods are disclosed herein for increasing the number of stem cells or precursor cells in vivo and in vitro. In several embodiments, methods are disclosed herein for increasing the number of neuronal stem cells or neuronal precursor cells. The methods include increasing the survival and/or proliferation of stem cells and/or precursor cells.

The methods include contacting stem cells or precursor cells, such as neuronal precursor cells and/or neuronal stem cells, with an effective amount of (1) a Notch ligand and (2) a growth factor and (3) angiopoietin-2, thereby increasing the survival and proliferation of the cells. In one specific non-limiting example, the method includes contacting the cells of interest with an effective amount of (1) Delta, (2) a growth factor, and (3) angiopoientin-2. In an additional non-limiting example, the method includes contacting the cells of interest with an effective amount of (1) a Notch ligand, (2) insulin, and (3) angiopoientin-2. In a further non-limiting example, the method the method includes contacting the cells of interest with an effective amount of (1) Delta, (2) insulin, and (3) angiopoientin-2. The methods can also include contacting the cells of interest with an effective amount Jak inhibitor. The cells can be any mammalian cells, including but not limited to primate cells such as human cells, and murine cells such as mouse or rat cells.

In one embodiment, stem cells and/or precursor cells are contacted in vitro with an effective amount of the Notch ligand, growth factor, and angiopoietin-2, such as, but not limited to Delta, insulin and angiopoietin-2. Generally, the Notch ligand, growth factor and angiopoietin-2 are included in a physiologically acceptable carrier, such as a tissue culture media or balanced salt solution and introduced into the cultured cells. As noted above, the cell can be any stem cell or precursor cell that can be propagated in vitro, such as, but not limited to, embryonic stem cells, neuronal stem cells and/or neuronal precursor cells. Stem cells and precursor cells contacted in vitro with an effective amount of the Notch ligand, growth factor, and angiopoietin-2, such as, but not limited to Delta, insulin and angiopoietin-2 can be maintained in vitro. Alternatively, stem cells and/or precursor cells can be contacted ex vivo with an effective amount of the Notch ligand, growth factor, and angiopoietin-2, such as, but not limited to Delta, insulin and angiopoietin-2 and then transplanted into a subject of interest. The subject of interest can be treated with a therapeutically effective amount of the Notch ligand, growth factor, and angiopoietin-2, such as, but not limited to Delta, insulin and angiopoietin-2, or can not be treated with a therapeutically effective amount of the Notch ligand, growth factor, and angiopoietin-2, such as, but not limited to Delta, insulin and angiopoietin-2.

In other embodiments, the disclosed methods include in vivo uses. Thus, the methods include selecting a subject of interest, and contacting stem cells or precursor cells in the subject with a therapeutically effective amount of (1) a Notch ligand, (2) a growth factor, and (3) angiopoientin-2. Suitable subjects include those subjects that would benefit from proliferation of stem cells or precursor cells, such as neuronal precursor cells or neuronal stem cells.

For example, the subject can have a neurodegenerative disorder or have a severed spinal cord. Thus the method can include selecting a subject with a neurodegenerative disorder or a spinal cord injury. Specific, non-limiting examples of a neurodegenerative disorder are Alzheimer's disease, Pantothenate kinase associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis. Suitable subject also include those subjects that are aged, such as individuals who are at least about 65, at least about 70, at least about 75, at least about 80 or at least about 85 years of age. In additional examples, the subject can have a spinal cord injury, Batten's disease or spina bifida. In further examples, the subject can have hearing loss, such as a subject who is deaf, or can be in need of the proliferation of proliferation of stem cells from the inner ear to prevent hearing loss.

The methods can also be used in association with procedures such as a surgical nerve graft, or other implantation of neurological tissue, to promote healing of the graft or implant, and promote incorporation of the graft or implant into adjacent tissue, such as for the treatment of spinal cord injury. According to another aspect, the compositions could be coated or otherwise incorporated into a device or biomechanical structure designed to promote nerve regeneration. In additional embodiments, spinal cord cells are treated with a therapeutically effective amount of a (1) Notch ligand (2) a growth factor, and (3) angiopoientin-2 in vitro, such as treatment with a therapeutically effective amount of (1) Delta, (2) insulin, and (3) angiopoiein-2. A therapeutically effective amount of the cells is then transplanted into a subject of interest, such as a subject with a spinal cord injury or spina bifida.

For use in vivo, the administration can be systemic or local. In one specific, non-limiting example, the therapeutically effective amount of a (1) Notch ligand (2) a growth factor, and (3) angiopoientin-2, such as treatment with a therapeutically effective amount of (1) Delta, (2) insulin, and (3) angiopoiein-2 is administered by injection into a ventricle of the central nervous system and/or administration into the spinal cord. In several embodiments, any local administration can used, such as administration into the cerebral spinal fluid. A therapeutically effective amount of a Jak inhibitor can also be administered locally to the subject.

In one non-limiting example, the method includes intraventricular infusion of a therapeutically effective amount of a Notch ligand, a therapeutically effective amount of a growth factor, and a therapeutically effective amount of angiopoietin-2, and optionally a therapeutically effective amount of a Jak inhibitor, into the central nervous system. Infusion can also be infused into the cerebral spinal fluid or by interstitial delivery to the central nervous system. For example, the agents can be introduced using a cannula and an osmotic pump. The Notch ligand, the growth factor, the angoipoitin-2, and optionally the Jak inhibitor, can be infused intraventricularly using an Ommaya reservoir, a plastic reservoir implanted subcutaneously in the scalp and connected to the ventricles within the brain by an outlet catheter. Solutions can be subcutaneously injected into the implanted reservoir and delivered to the ventricles by manual compression of the reservoir through the scalp. Several implantable pumps have been developed that possess several advantages over the Ommaya reservoir. These can be implanted subcutaneously and refilled by subcutaneous injection and are capable of delivering drugs as a constant infusion over an extended period of time. Furthermore, the rate of drug delivery can be varied using external handheld computer control units.

Compositions including a therapeutic moiety, such as, but not limited to, a therapeutically effective amount of a (1) Notch ligand (2) a growth factor, and (3) angiopoientin-2, such as treatment with a therapeutically effective amount of (1) Delta, (2) insulin and/or GDNF, and (3) angiopoiein-2, can be delivered by way of other types of pumps (see Langer, supra; Sefton, CRC Crit. Ref. Biomed. Eng. 14:201, 1987; Buchwald et al., Surgery 88:507, 1980; Saudek et al., N Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249:1527-33, 1990).

In one example, a pump is implanted (for example see U.S. Pat. Nos. 6,436,091; 5,939,380; and 5,993,414). Implantable drug infusion devices are used to provide patients with a constant and long-term dosage or infusion of a therapeutic agent. Such device can be categorized as either active or passive.

Active drug or programmable infusion devices feature a pump or a metering system to deliver the agent into the patient's system. An example of such an active infusion device currently available is the Medtronic SYNCHROMED™ programmable pump. Passive infusion devices, in contrast, do not feature a pump, but rather rely upon a pressurized drug reservoir to deliver the agent of interest. An example of such a device includes the Medtronic ISOMED™.

In particular examples, the methods include administering the therapeutic agents by sustained-release systems. Suitable examples of sustained-release systems include suitable polymeric materials (such as, semi-permeable polymer matrices in the form of shaped articles, for example films, or microcapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al., Biopolymers 22:547-556, 1983, poly(2-hydroxyethyl methacrylate)); (Langer et al., J. Biomed. Mater. Res. 15:167-277, 1981; Langer, Chem. Tech. 12:98-105, 1982, ethylene vinyl acetate (Langer et al., Id.) or poly-D-(−)-3-hydroxybutyric acid (EP 133,988).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (for example, U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

As noted above, for use in any of the therapeutic methods disclosed herein, administration of the a therapeutically effective amount of a (1) Notch ligand (2) a growth factor, and (3) angiopoientin-2, such as treatment with a therapeutically effective amount of (1) Delta, (2) insulin and/or GDNF, and (3) angiopoiein-2 (and optionally additional agents, such as a Jak inhibitor) can be systemic. Oral, intravenous, intra-arterial, subcutaneous, intra-peritoneal, intra-muscular, intra-ventricular, intra-nasal transmucosal, subcutaneous, topical and even rectal administration is contemplated.

Pharmacological compositions for use can be formulated in a conventional manner using one or more pharmacologically (for example, physiologically or pharmaceutically) acceptable carriers comprising excipients, as well as optional auxiliaries that facilitate processing of the active compounds into preparations which can be used pharmaceutically. Proper formulation is dependent upon the route of administration chosen.

Thus, for injection, the active ingredient can be formulated in aqueous solutions, preferably in physiologically compatible buffers. For transmucosal administration, penetrants appropriate to the barrier to be permeated are used in the formulation. Such penetrants are generally known in the art. For oral administration, the active ingredient can be combined with carriers suitable for inclusion into tablets, pills, dragees, capsules, liquids, gels, syrups, slurries, suspensions and the like. Formulations can also be prepared for use in inhalation therapy. For administration by inhalation, the active ingredient is conveniently delivered in the form of an aerosol spray presentation from pressurized packs or a nebuliser, with the use of a suitable propellant.

The therapeutically effective amount of a (1) Notch ligand (2) a growth factor, and (3) angiopoientin-2, such as treatment with a therapeutically effective amount of (1) Delta, (2) insulin, and (3) angiopoiein-2, optionally with a Jak inhibitor, can be formulated for parenteral administration by injection, such as by bolus injection (a pulsatile dose) or continuous infusion. Similarly, a therapeutically effective amount of a (1) Notch ligand (2) a growth factor, and (3) angiopoientin-2, such as treatment with a therapeutically effective amount of (1) Delta, (2) insulin, and (3) angiopoiein-2 (and optionally a Jak inhibitor), can be formulated for intratracheal or for inhalation. Such compositions can take such forms as suspensions, solutions or emulsions in oily or aqueous vehicles, and can contain formulatory agents such as suspending, stabilizing and/or dispersing agents. Other pharmacological excipients are known in the art.

Therapeutically effective doses of the presently described compounds can be determined by one of skill in the art, with a goal of achieving a desired number of stem cells and/or precursor cells, such as neuronal stem cells and/or neuronal precursor cells. An increase in the number of stem cells and precursor cells can be assessed using markers of these cells, or by determining an increase in the number of differentiated progeny of these cells. Thus, in several examples, for confirming increased neuronal stem cell or neuronal progenitor cell survival, an increase in the number of differentiated neuronal cells (such as dopaminergic cells) can be detected. Method for measuring increased numbers of differentiated cells are known in the art. For example, to detect dopamineric cells, increased numbers of cells that express tyrosine hyroxylase or dopamine decarboxylase can be detected, such as by using immunohistochemistry. However, other measures, such as behavioral assessments or electrophysiological techniques can also be utilized. One of skill in the art can readily detect an increase in the number of cells of a specific phenotype.

The relative toxicities of the compounds make it possible to administer in various dosage ranges. In one example, the compound is administered orally in single or divided doses. The specific dose level and frequency of dosage for any particular subject may be varied and will depend upon a variety of factors, including the activity of the specific compound, the extent of existing disease activity, the age, body weight, general health, sex, diet, mode and time of administration, rate of excretion, drug combination, and severity of the condition of the host undergoing therapy.

### Screening Methods

A method is provided herein for identifying an agent that alters the survival or proliferation of neuronal stem cells. The method includes contacting a neuronal stem cell with an effective amount of an agent of interest. The expression of Tie-2 and/or the binding of cholera toxin subunit B is determined. Methods are provided for identifying an agent that increases the proliferation and/or survival of neuronal stem cells. Increased expression of Tie-2, without an increase in the binding of cholera toxin B in the neuronal stem cell indicates that the agent increases the survival or proliferation of neuronal stem cells. The method can also include assessing the expression of Hes-3. An increase in the expression of Tie-2 without an increase in the binding of Cholera toxin B, with an increase in expression of Hes-3 in the stem cells contacted with the agent indicates that the agent is of use to increase the survival or proliferation of neuronal stem cells. The cell can be any stem cell of interest, such as a dopaminergic stem cell. In additional examples, the cell is any mammalian cell, such as a human cell. In additional examples, the cell expresses sonic hedgehog.

Methods are also provided herein for identifying an agent that increases differentiation of neuronal cells. Decreased expression of Tie-2 indicates that the agent increases the differentiation of stem cells and/or precursor cells. Decreased expression of Tie-2, with an increase in the binding of cholera toxin B in the neuronal stem cell indicates that the agent increases the differentiation of neuronal stem cells. The method can also include assessing the expression of Hes-3. An decrease in the expression of Tie-2 with an increase in the binding of Cholera toxin B, with an decrease in expression of Hes-3 in the neuronal stem cells contacted with the agent indicates that the agent is of use to increase the differentiation of neuronal stem.

The method provided herein can include comparing the expression of Tie-2, Hes-3 and/or the binding of cholera toxin to a control, such as a cell not contacted with the agent, a cell contacted with vehicle alone, or a standard value.

The test compound can be any compound of interest, including chemical compounds, small molecules, polypeptides, growth factors, cytokines, or other biological agents (for example antibodies). In several examples, a panel of potential neurotrophic agents are screened. In other embodiments a panel of polypeptide variants is screened.

Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991), a peptide library (U.S. Pat. No. 5,264,563); a peptidomimetic library (Blondelle et al., Trends Anal Chem. 14:83-92, 1995); a nucleic acid library (O'Connell et al., Proc. Natl Acad. Sci., USA 93:5883-5887, 1996; Tuerk and Gold, Science 249:505-510, 1990; Gold et al., Ann. Rev. Biochem. 64:763-797, 1995); an oligosaccharide library (York et al., Carb. Res. 285:99-128, 1996; Liang et al., Science 274:1520-1522, 1996; Ding et al., Adv. Expt. Med. Biol. 376:261-269, 1995); a lipoprotein library (de Kruif et al., FEBS Lett. 3 99:23 2-23 6, 1996); a glycoprotein or glycolipid library (Karaoglu et al., J Cell Biol. 130.567-577, 1995); or a chemical library containing, for example, drugs or other pharmaceutical agents (Gordon et al., J Med. Chem. 37.1385-1401, 1994; Ecker and Crooke, BioTechnology 13:351-360, 1995). Polynucleotides can be particularly useful as agents that can alter a function of stem cells (such as, but not limited to ES cells and neuronal stem cells) and precursor cells because nucleic acid molecules having binding specificity for cellular targets, including cellular polypeptides, exist naturally, and because synthetic molecules having such specificity can be readily prepared and identified (see, for example, U.S. Pat. No. 5,750,342).

In one embodiment, for a high throughput format, neuronal stem cells can be introduced into wells of a multiwell plate or of a glass slide or microchip, and can be contacted with the test agent. Generally, the cells are organized in an array, particularly an addressable array, such that robotics conveniently can be used for manipulating the cells and solutions and for monitoring the stem or precursor cells, particularly with respect to the function being examined. An advantage of using a high throughput format is that a number of test agents can be examined in parallel, and, if desired, control reactions also can be run under identical conditions as the test conditions. As such, the methods disclosed herein provide a means to screen one, a few, or a large number of test agents in order to identify an agent that can alter a function of cells, for example, an agent that induces the cells to differentiate into a desired cell type, or that affects differentiation, survival and/or cell proliferation.

The disclosure is illustrated by the following non-limiting Examples.

### EXAMPLES

An ultimate goal of regenerative medicine is the replacement of compromised cells such as neurons in the diseased adult brain. Cell transplantation is a promising avenue, and the adult brain possesses regenerative capacity by activation of quiescent, resident stem cells (see, for example Arvidsson et al., Nat Med 8, 963-70, 2002; Magavi et al., Nature 405, 951-5, 2000). Although this process is inherently inefficient, pharmacological manipulation can significantly enhance it and induce behavioural recovery (Androutsellis-Theotokis et al., Nature 442, 823-6; 2006; Nakatomi et al., Cell 110, 429-41, 2002) without the problems of immuno-compatibility and controlled differentiation'associated with grafting. Such manipulations have been shown to confer significant functional recovery in the injured brain even without replenishment of lost neurons (Androutsellis-Theotokis, A. et al, supra). The identification of novel targets for manipulation, therefore, is essential.

A concept that has been arising, therefore, is that activation of the stem cell/progenitor niche in the adult brain may be beneficial also via neuro-protective functions mediated by trophic factors released by the activated progenitor cells (Ourednik, et al., Nat Biotechnol 20, 1103-10, 2002). Neuronal stem cells (NSCs) reside in the neurovascular niche, a local microenvironment that comprises the stem cell, the vasculature, and mature neurons; interactions between all combinations of these cells determine the state of the niche (Monje et al., Nat Med 8, 955-62, 2002; Shen, Q. et al., Science 304, 1338-40, 2004; Kempermann et al., Curr Opin Neurobiol 14, 186-91, 2004). It is difficult to conceive, therefore, that one can target one cell type in the niche without affecting the others. In addition, factors that can activate NSCs need to be identified. If the appropriate signals are identified, factors that influence these pathways can be used to induce their long-term activation and induce neuroprotection, resulting the treatment of neurodegenerative diseases such as Parkinson's.

It is disclosed herein that the adult central nervous system (and human tumours) is diffusely rich in peri-vascular neural stem cells, identified by the expression of the transcription factor Hairy and enhancer of split 3 (Hes3) and can be activated by single intraventricular injections of signals that affect the vasculature such as Angiopoietin-2, insulin, and Delta-like 4. In the 6-hydroxy-dopamine model of Parkinson's disease, activation was followed by extensive neuroprotection of nigro-striatal dopaminergic neurons and behavioural recovery, as assessed by amphetamine-induced rotometry. Methods are disclosed herein that can be used to activate and assess the endogenous stem cell compartment with direct relevance to regenerative medicine, stem cell activation and neuroprotection. Methods are also disclosed herein for the treatment of an exemplary neurodegenerative disorder, namely Parkinson's disease.

The studies presented herein demonstrate that activation of the stem cell/progenitor (precursor) niche in the adult brain is be beneficial also via neuro-protective functions. Neuronal stem cells (NSCs) reside in the neurovascular niche, a local microenvironment that comprises the stem cell, the vasculature, and mature neurons; interactions between all combinations of these cells determine the state of the niche. In the experiments described below, Hes3 immuno-reactivity was used to identify a wide-spread peri-vascular NSC niche throughout the parenchyma and spinal cord. Vascular signals were used to pharmacologically activate the endogenous stem cell niche. Insulin, Ang-2 and Delta 4 (Dll4) were used to allow NSC activation. These agents (insulin, Ang-2 and Dll4) can be used with an inhibitor of JAK kinase. The experimental results demonstrate that single injections of the appropriate vascular signals induce long-term activation, neuroprotection, and lasting behavioural recovery in a model of Parkinson's disease.

### Example 1

### Materials and Methods

Cell culture: E13.5 cortical embryonic mouse CNS stem cells were grown as previously described (Johe, et al., Genes Dev 10, 3129-40, 1996). Cells were expanded in serum-free DMEM/F12 medium with N2 supplement and FGF2 (20 ng/ml) for 5 days under 5% oxygen conditions and were re-plated fresh or from frozen stocks at 1,000-10,000 cells per cm². FGF2 was included throughout the studies, unless otherwise stated. Adult rat (3-6 months old) or adult mouse (2-4 months old) SVZ NSC cultures were grown in the same medium as the fetal cultures.

Magnetic affinity cell sorting for AC133 (prominin-1): Dissociated E13.5 murine cortical tissue was resuspended in 300 ml of N2 containing medium without FGF2 or PDGF-AA. Thirty milliliters (ml) of prominin-coated beads (Miltenyi Biotech, Auburn, Calif.) were mixed with approx. 6-10 million cortical cells and incubated for 40 minutes at 6° C. At the end of incubation, the cell suspension was placed on MS columns (Miltenyi Biotech) in the presence of a magnetic field. AC133-cells were eluted by washing twice with 1 ml N2 medium. The column was removed from the magnet and AC133+ cells were eluted by washing with N2 into a separate tube.

Pharmacological treatments: Unless otherwise stated, the following concentrations were used: Dll4 and Ang-2 (in vitro, 500 ng/ml; in vivo 12.5 μg as a single injection), FGF2 (in vitro, 20 ng/ml), JAK inhibitor (in vitro, 200 nM; in vivo, 250 ng as a single injection), insulin (in vitro, 25 μg/ml; in vivo, 40 μg as a single injection), DAPT (1 μM), Fluorogold (0.5 μg). For the survival assays, compounds were added daily and Notch ligands every 2 days for the duration of the experiment. For the effects of kinase inhibitors on Notch activation, cells were pre-incubated for 1 h prior to Notch activation.

3D image analysis: Three-dimensional images were generated by employing Volocity 3D imaging software by Improvision on confocal z-stacks.

Blood vessel counting and area quantitation: Zeiss software was used to identify, count, and measure blood vessels stained for the pan-endothelial marker RECA-1. To measure the average distance between adjacent Hes3+ cells along the same blood vessel, 25 high-power (×63) confocal z-stack images were used from brain sections stained for endothelial markers and Hes3.

In vivo studies: Male adult (3-6 months) Sprague Dawley rats (Charles River Laboratories), weighing 250-350 gm, were used for pharmacological treatments and lesion experiments.

Surgeries:

6OHDA Lesion. Some animals underwent unilateral lesion of the nigrostriatal dopamine pathway by stereotactical injection of 50 μg 6-hydroxydopamine (6-OHDA) into the right striatum, using the following coordinates: bregma AP +0.5 mm, ML −3.0 mm, VD +5.5 mm.

Pharmacological treatments. Unlesioned animals, or animals two weeks after 6-OHDA lesion, were treated with different pharmacologic agents. Five microliters (μl) of different drugs were stereotactically injected into the right lateral ventricle using the following stereotaxic coordinates: bregma AP −0.9 mm, ML −1.4 mm, VD +3.8 mm. The following reagents were used: Dll4 (2 mg/ml), Ang-2 (1 mg/ml), insulin (8 mg/ml), Jak-Inhibitor (20 μM), either alone or in combination.

After all surgeries, animals were allowed to recover from the anesthesia and were put back into their home cages, where they were given access to food and water ad libitum.

Labeling of dividing cells: Animals received intraperitoneal injection of the tracer Bromodeoxyuridine (BrdU) (50 mg/kg) every 12 hours for 5 days beginning on day 1 post-op to label dividing cells.

Amphetamine induced rotations: At multiple time points over the course of the study, lesioned rats were injected intra-peritoneally with D-Amphetamine sulfate (5 mg/kg) and placed in automated rotometer bowls. Rotational behavior was assessed by monitoring total number of whole body (360°) turns over 60 min.

Immunohistochemistry: Under deep anesthesia, animals were perfused transcardially with a rinse of saline, followed by 4% formaldehyde fixative (pH 7.4). Brains were removed immediately, stored in the fixative solution overnight and then in 30% sucrose for 3 days. Brains were frozen-sectioned at 16 μm. Immunohistochemical detection of BrdU and Hes3 was performed with an antigen-retrieval step (sections were boiled in 0.02M citrate, pH 6.0 in a microwave for 5 min, washed 3 times with distilled water, and incubated for 45 min in 2M HCl, at room temperature).

MRI Imaging

MnCl₂ administration: Adult male Sprague-Dawley rats received stereotactical injections of 6-OHDA into the right striatum, and saline injections of equal volume into the left striatum as a negative control. In order to use Mn²⁺ as a contrast agent (Lee et al., Curr Pharm Biotechnol 5, 529-37, 2004), rats were injected intraperitoneally with 30 mg/kg MnCl₂ 24 hrs prior to each imaging session; during neuronal tract tracing, 100 mM MnCl₂ was administered into the substantia nigra 2 wks after 6-OHDA lesioning.

MRI Method: MRI was performed 24 hrs after the lesion, followed by weekly MRI over 7 weeks. Rats were anaesthetized with 1.5% isofluorane and placed prone in a stereotaxic holder with brain centered in a 72/25 mm volume transmit/surface receive coil ensemble. Body core temperature was maintained at 37° C. with warm air flow over the rat. A pressure transducer was used to monitor the respiration cycle. MR imaging was performed on a 21 cm horizontal bore 7 Tesla scanner operating on a Bruker Avance platform (Bruker Biospin Inc. Billerica, Mass.).

Three mutually perpendicular slice images through the brain were acquired as scout images. Fourteen contiguous 1 mm thick axial images (slice number 6-7 centered at the Bregma) of the brain were acquired using a fast spin echo (FSE) sequence (matrix 256², NA=8, echoes=8, TR/TE=3500/14.15 ms, Field of View [FOV]=3.2 cm); an identical set of FSE images with T₁ weighting (TR/TE=355/10.25 ms) was also acquired. In order to obtain quantitative T₂ values, T₂ weighted axial images (TR/TE=3500/20 ms, 8 echoes, FOV=3.2 cm, matrix 128²) were acquired. A 2-D multi slice look-locker sequence with 20 point samples along the inversion recovery curve was used to calculate T₁ (TE/TR=2.2/10⁴ ms, flip angle=25°, FOV=3.2 cm, Matrix 128, slice thickness=1 mm, inversion interval=400 ms).

Quantitative T₁ and T₂ maps were calculated using custom written software in MATLAB (Mathworks Inc., Natick, Mass.) taking into account the perturbation caused by flip angle in determining T₁.

Reagents: The following reagents and antibodies were used in the studies described below: FGF2 (233-FB), mouse Dll4 (1389-D4), CNTF (577-NT), Fibronectin (1030-FN), human angiopoietin-1 (923-AN), human angiopoietin-2 (623-AN), mouse Tie-2/Fc (762-T2), from R&D; JAK Inhibitor I (420099), DAPT (565770), SB203580 (559389), from Calbiochem; Polyornithine (Sigma, P-3655), ECL reagents (Pierce, 34080), polyacrylamide gradient gels (Invitrogen), BrdU (Boehringer, 84447723), Fluorogold from Fluorochrome, LLC, insulin (Sigma, 19278), Alexa-Fluor-conjugated secondary antibodies (Molecular Probes), HRP-conjugated secondary antibodies (Jackson Immunoresearch), DAPI (Sigma, D-8417), Vectastain ABC kit (Vector, PK-6101), Peroxidase substrate kit (Vector, SK-4100), and general chemicals from Sigma.

For immunohistochemical staining and Western blotting, antibodies against the following markers were used: Notch1IC (MAB5352) and GDNF (AB5252P) from Chemicon; SSEA1 (Developmental Studies Hybridoma Bank), human nestin (Chemicon, MAB5326), nestin (Chemicon, MAB353), Tuj1 (Covance, MMS-435P), GFAP (Dako, z0334 and Chemicon, MAB360), CNPase (Chemicon, MAB326), BrdU (Accurate, H5903), Sox2 (R&D, MAB2018); pSer473-Akt (92715), pThr308-Akt (9275), pSer2448-mTOR (29715), p38 (9212), pThr180/Tyr182-p38 (9211), pIGFR1/InsulinR (3024, 3021), from Cell Signaling; STAT3 (482), pSer727-STAT3 (8001-R), pTyr705-STAT3 (7993), Akt (5298), Sonic Hedgehog (1194), Hes3 (25393), Ang-1 (sc-6319), Ang-2 (sc-7015), Tie-2 (sc-324), Tie-2 (sc-31266) from Santa Cruz; α-tubulin (Sigma, T-6074); tyrosine hydroxylase (P80101 and P40101 from Pel-Freez), AC133 (Miltenyi, 130-090-422); PDGFRα (E8664) from Spring Bioscience; RECA-1 (MCA 970GA) from Serotec; pTie-2 (Af2720) from RnD Systems.

Statistical analysis: In all studies, mean±standard deviation (SD) or standard error from the mean (SEM) are presented as stated. Asterisks identify experimental groups significantly different (p value, 0.05) from control groups by the Student's T-test (Microsoft Excel), with a Bonferroni correction for multiple comparisons (alpha value, 0.05), where applicable.

### Example 2

### Hes3 Mediates NSC Survival

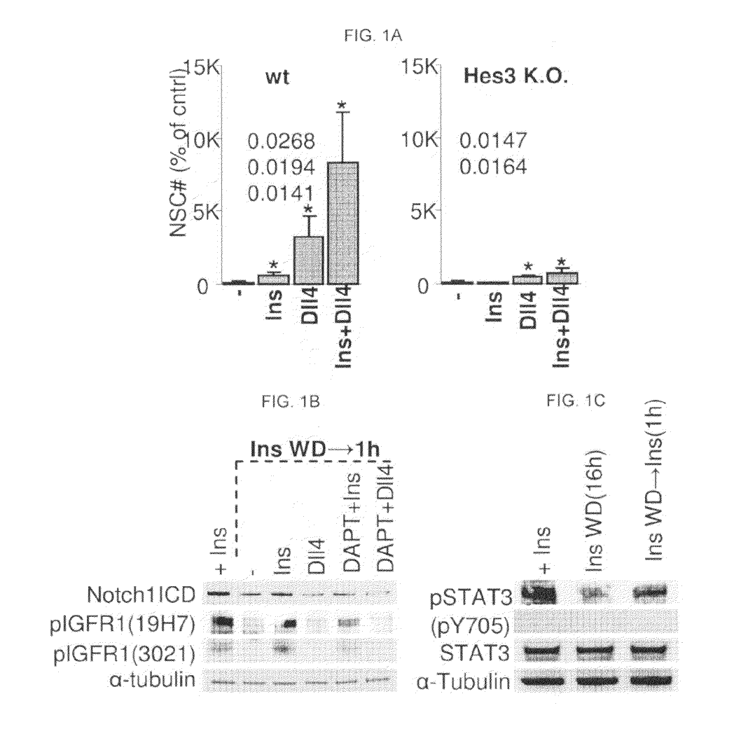

Hes3 mRNA is present in fetal NSC cultures under conditions that support their self-renewal but CNTF-induced differentiation (Bonni, A. et al., Science 278, 477-83, 1997; Rajan & McKay, J Neurosci 18, 3620-9, 1998): downregulates Hes3 message (Androutsellis-Theotokis, A. et al., Nature 442, 823-6, 2006). Knockout studies have shown a role of Hes3 in self-renewal (Hatakeyama, J. et al., Development 131, 5539-50, 2004). It was investigated if Hes3 mediates stem cell functions in vitro to assess its use as a functional NSC marker. Fetal (E13.5) cortical tissue was dissociated and magnetic sorting was used to isolate a cell population that expressed AC133, an established marker of NSCs (Uchida, N. et al., Proc Natl Acad Sci USA 97, 14720-5, 2000). Sorted (AC133+) cells expressed Hes3 and Sox2, another established NSC marker (Zappone, M. V. et al., Development 127, 2367-82, 2000). Immuno-staining showed that Hes3 protein is expressed in self-renewing fetal NSC cultures, maintained when treated with the Notch ligand Dll4 for 2 days (a treatment that supports NSC properties and promotes their survival in vitro), but is lost when the cells are differentiated by a 2 day CNTF treatment. To probe potentially therapeutic uses of differentiated NSCs cells, it was found that in contrast to self-renewing NSCs, they express glial derived neurotrophic factor (GDNF) when bFGF is withdrawn for 5 days, a growth factor known to promote the survival of dopaminergic neurons (Lin et al., Science 260, 1130-2, 1993). To investigate if Hes3 is a mediator of pro-survival signals for NSCs, adult mouse SVZ cultures (which respond strongly to pro-survival signals) were treated with insulin, Dll4, and a combination of the two. Wild-type cells responded by a great increase in colony number and overall cell number, whereas cells from Hes3 null mice only showed moderate (but significant) benefits (FIG. 1a). These data demonstrate that Hes3 partly mediates the pro-survival functions of insulin and Notch.

It was previously shown that in NSCs Notch activation leads to rapid phosphorylation of Akt (Androutsellis-Theotokis, A. et al., Nature 442, 823-6, 2006), a mediator of insulin signalling (Cantley, Science 296, 1655-7, 2002). The rapid integration of the Notch and insulin signals to Akt and Hes3 suggests that these pathways may converge at the level of receptor activation. Insulin treatment of the NSC cultures induced cleavage (activation) of the Notch receptor (FIG. 1b). Insulin withdrawal for 2 days reduced the levels of cleaved Notch, and acute (1 h) insulin treatment induced Notch activation. This result suggests that insulin facilitates Notch activation by endogenous or exogenous Notch ligands. Activation of the Notch receptor by addition of insulin or Dll4 was inhibited by DAPT, a γ-secretase inhibitor that blocks Notch cleavage. Activation of the insulin/IGF-1 receptors by insulin treatment was partly blocked by DAPT. Insulin maintained STAT3-Ser727 phosphorylation levels, and acutely (1-h) induced it following a 16-h insulin withdrawal phase (FIG. 1c). These results demonstrate cross-talk between the insulin and Notch pathways, integration at the level of second messenger activation, and that their downstream mediator Hes3 is a marker of NSCs.

### Example 3

### Angiopoietin Balance in NSC Cultures

Endothelial cells of the vasculature are a source of Dll4 in fetal and adult tissues (Gale, N. W. et al. Haploinsufficiency of delta-like 4 ligand results in embryonic lethality due to major defects in arterial and vascular development. Proc Natl Acad Sci USA 101, 15949-54 (2004); Duarte, A. et al., Genes Dev 18, 2474-8, 2004), and NSCs likely utilize Dll4 to ensure their survival as they associate closely with the vasculature. It was investigated whether other vascular signals also promote NSC survival in vitro.

Angiopoietin-1 (Ang-1) is expressed in osteoblasts of the bone marrow and promotes the quiescence of the haematopoietic stem cell niche (Arai, F. et al., Cell 118, 149-61, 2004). Ang-1 is also expressed in pericytes where it regulates angiogenesis (Suri, C. et al., Cell 87, 1171-80, 1996) and may be a homing signal for young neurons (Ohab et al., J Neurosci 26, 13007-16, 2006). It was hypothesized that Ang-1 antagonism could activate the endogenous stem cell niche by forcing them to exit quiescence. Furthermore, Ang-1 activates p38 MAP kinase (Harfouche, R. et al., Faseb J 17, 1523-5, 200; Zhu et al., J Vasc Res 40, 140-8, 2003), a kinase that opposes NSC survival⁴ and adult cardiomyocyte self-renewal (Engel, F. B. et al., Genes Dev., 2000). Angiopoietin-2 (Ang-2) opposes actions of Ang-1 (Maisonpierre, P. C. et al., Science 277, 55-60, 1997) It was Ang-2 could activate of endogenous NSCs.

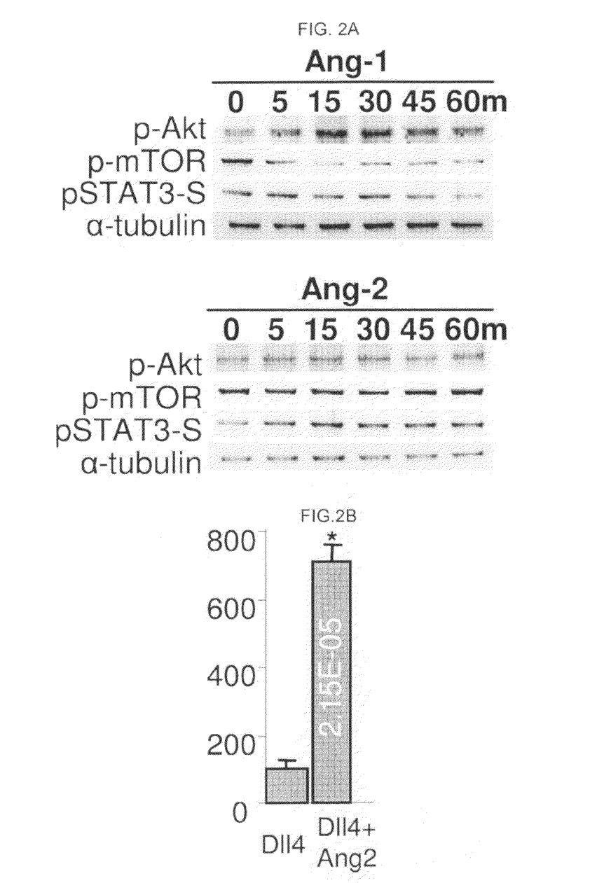

Fetal NSCs in culture express Ang-1, and treatment (2 days) with Dll4 maintained it whereas CNTF reduced it. Ang-2 expression was maintained under these conditions. This result suggests that NSC cultures may utilize the angiopoietins for cellular functions. Ang-2 induced mTOR and STAT3-S727 phosphorylation, unlike Ang-1 (FIG. 2a). Ang-1, however, induced Akt phosphorylation. These results suggest that STAT3-S727 phosphorylation is a common integrator of pro-survival actions and that it can be regulated independently of Akt. Ang-2 profoundly increased the generation of adult rat SVZ NSCs in vitro (FIG. 2b). In cultures treated with Dll4, a factor that stimulates their survival (Androutsellis-Theotokis, A. et al., Nature 442, 823-6, 2006), Ang-2 significantly increased colony formation and overall cell numbers. These data suggest that multiple vascular cytokines act co-operatively to promote the survival of NSCs.

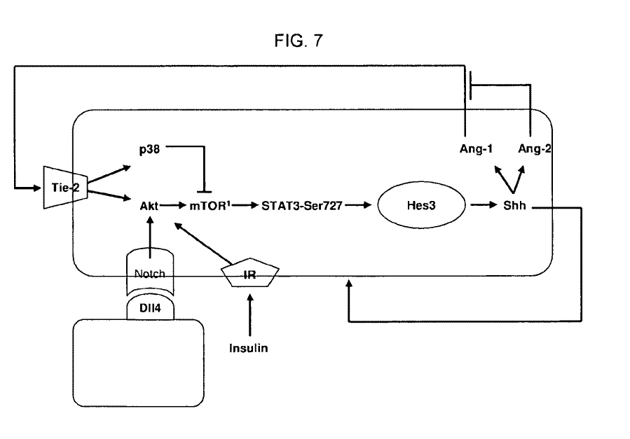

These results suggest a model whereby Notch, insulin, and Ang-2 promote the generation of NSCs, whereas Ang-1 promotes their quiescence by opposing STAT3-S727 phosphorylation and survival through Akt phosphorylation. Sonic hedgehog (Shh) is a cytokine with established mitogenic properties, and can be induced by Hes3. Shh also regulates the expression of angiopoietins, providing feedback control of this pathway (FIG. 7). These results suggest a conserved mechanism that maintains quiescence of stem cells in the blood and brain.

### Example 4

### Hes3 Identifies the Parenchymal NSC Niche

Hes3+ cells in the central nervous system (CNS) parenchyma were characterized and the means to activate the NSC compartment in vivo was investigated. Single injections of pharmacologically active compounds were performed in the lateral ventricle, bromodeoxyuridine (BrdU) was administered intraperitoneally (days 2-4) and the animals were sacrificed on day 5. At the subventricular zone (SVZ), a single intra-ventricular injection of Dll4 induced STAT3-S727 phosphorylation and Hes3 expression within 5 days. Single intra-ventricular injections of Dll4, insulin, a combination of Dll4 and insulin, Ang-2 or a mix containing Dll4, insulin, Ang-2 and a JAK kinase inhibitor increased the number of proliferating cells in the SVZ. Because the strongest activation was achieved with the mix, it was used to assess activation in the peri-aqueductal area (midbrain), hippocampus, and spinal cord. BrdU+ cell numbers were elevated in all these areas (FIG. 3a).

To characterize the Hes3+ cell population in the adult CNS, sections were co-stained with Hes3 and other stem cell markers. In the SVZ, Hes3 was co-expressed with nestin, Sox2, and PDGFRα, which are markers of stem cells. Outside the SVZ, nestin and PDGFRα are not expressed in neural cells, suggesting that NSCs are found only in localized areas of the brain (the SVZ and SGZ), or that they are held in a quiescent state and express a different set of markers, rendering them difficult to identify. Hes3+ cells were found throughout the parenchyma of the brain. These cells were invariably associated with blood vessels, which in addition to typically vascular markers also express PDGFRα and nestin. A closer examination of the Hes3+ cell placement revealed a regular spacing along blood vessels at 28.2±10 μm. Hes3+ cells expressed glial fibrillar acidic protein (GFAP), another marker of adult NSCs, but represented only a small minority of peri-vascular GFAP+ cells. These data suggest a mechanism for the propagation of the NSC activation signal along the external surface of blood vessels that depends on secreted factors like Ang-2 and proximity between adjacent NSCs.

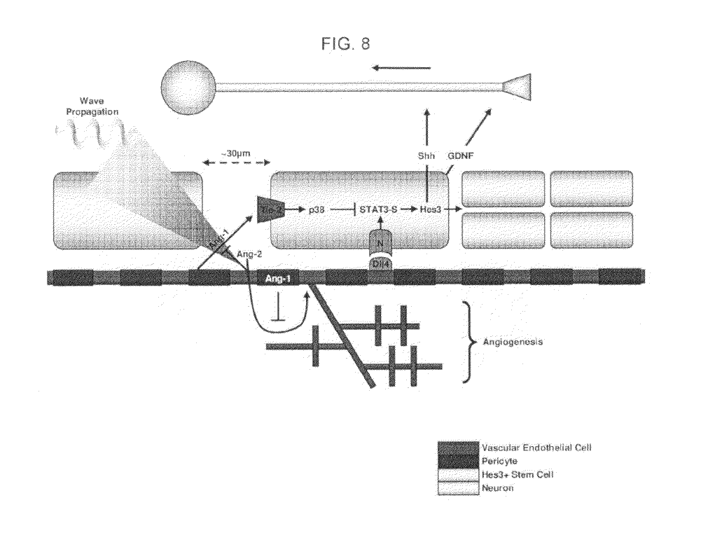

It was postulated that the angiopoietins are also important in regulating the quiescence/activation of the NSC niche. Corroborating this possibility is the fact that the SVZ has a lower ratio of Ang-1/Ang-2 than the brain parenchyma. The single injection treatments increased the incidence of Hes3+ cells associated with a blood vessel throughout the brain (FIG. 3b). These data show that vascular signals can be used to mobilize the endogenous NSC niche throughout the brain. In addition, these findings suggest a signalling model that can be sustained for long periods of time due to various positive feedback loops and can be propagated over long distances as several of the signals are secreted and Hes3+ cells are spatially arranged along blood vessels in an equidistant manner (FIG. 8).

### Example 5

### NSC Activation in the Midbrain

Activation of the NSC niche can lead to behavioural recovery from lesions such as stroke (see, for example, Nakatomi, H. et al., Cell 110, 429-41, 2002), raising the possibility that such treatments may affect the survival of mature neurons directly or indirectly. To investigate this possibility, the following studies concentrated on the adult midbrain peri-aqueductal area for a number of reasons. First, as part of the cerebro-ventricular system, it is easily accessible by injecting drugs in the lateral ventricles. Second, the aqueduct is lined with dopaminergic neurons, which are particularly sensitive to cytotoxic insults. Third, these neurons do not exhibit STAT3-Y705 phosphorylation (Bachtell et al., J Pharmacol Exp Ther 302, 516-24, 2002), suggesting that all STAT3 activity is mediated by phosphorylation on serine 727. In this respect, therefore, these mature neurons may have signal transduction requirements comparable to NSCs, which also exhibit no STAT3-Y705 phosphorylation. Finally, Shh, which is induced by Hes3 activation promotes the survival of TH+ neurons (see, for example, Rafuse et al., Neuroscience 131, 899-916, 2005). It is demonstrated herein that that Hes3+ NSCs that are induced to differentiate express GDNF, another neuroprotective agent for dopaminergic neurons, suggesting that endogenous NSCs can protect dopaminergic neurons during their self-renewing state (via Shh) and during their differentiation (via GDNF). Thus, treatments that induce serine 727 phosphorylation can be particularly effective on dopaminergic neurons.

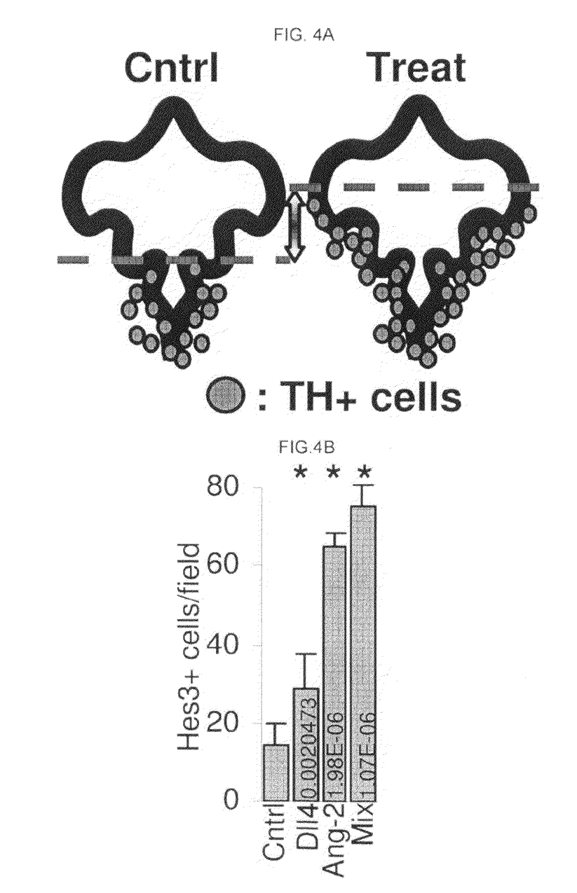

TH+ neurons were found around the aqueduct and in the substantia nigra are inter-mingled with Hes3+ cells, suggesting that mobilizing Hes3+ cells may have pronounced effects on adjacent TH+ neurons. A single intraventricular injection of the mix increased the number of BrdU+ cells around the aqueduct, demonstrating that this administration is a valid route to affect the midbrain. The injection also stimulated expression of Hes3 around the aqueduct but also in the midbrain parenchyma (FIG. 4b). Hes3+ cells in the midbrain parenchyma including the substantia nigra express Shh and some also express GDNF. Treatment with the mix also increased the circumference of the aqueduct that is lined by TH+ cells by 70% (FIG. 4a), suggesting that additional changes occur in the midbrain that alter the plasticity of the area around the aqueduct.

### Example 6

### Vascular Effects and Predictive Imaging

The compounds that used in the experiments described herein to activate endogenous NSCs have known effects on the vasculature. Progenitor cells in the SVZ receive dopaminergic inputs from nigrostriatal dopaminergic neurons (Hoglinger et al., Nat Neurosci 7, 726-35, 2004). It is possible that a compromised dopaminergic input to the progenitor/stem cell niche in the striatum affects NSC functions within the neurovascular niche and subsequently causes changes in the vascular properties around the affected area. This possibility was assessed with imaging techniques.

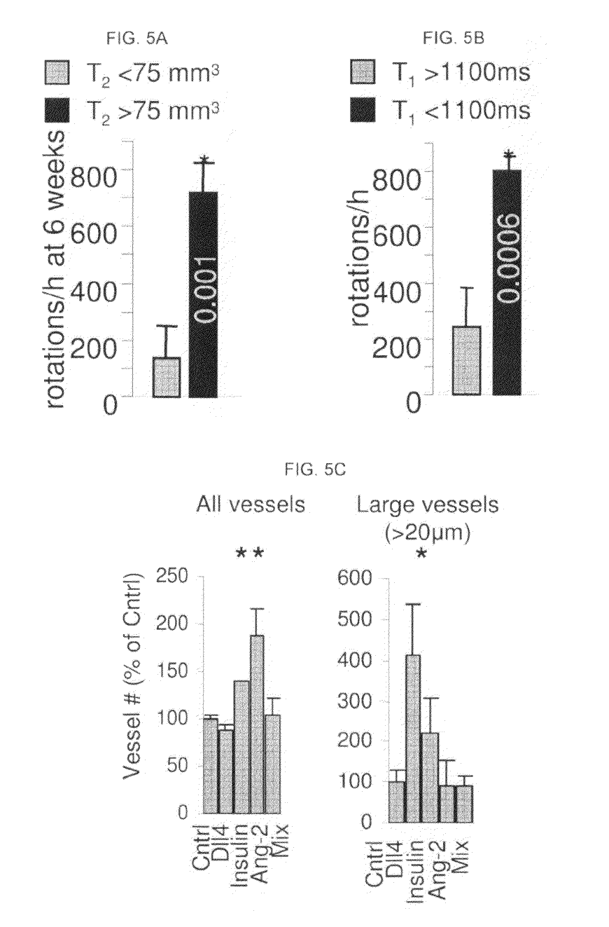

MRI imaging is non- or low-invasive technique to assess brain functions and pathology (Silva et al., NMR Biomed 17, 532-43, 2004). It is investigated if MRI imaging could be used at early time points following 6OHDA lesion to predict long-term behavioural effects. This approach could lead to a diagnostic research protocol and the collection of additional data from the experimental animals. A number of rats were lesioned and the T2 signal was measured (i.e., without injection of a contrast dye, so that the signal comes from the concentration of water) one day after. The data was split into two groups based on their T2 values (the differences were due to variability between individual animals. A T2 value of 75 mm³ was chosen to split the two groups roughly in the middle. Their behavioural improvement was then measured (amphetamine-induced rotations) and the value at 6 weeks was used to ask if T2 values (at day 1) and rotometry values (at week 6) correlated. Significant correlation was found between T2 and rotometry values (FIG. 5a). To enhance the sensitivity of MRI, a contrast agent such as manganese can be injected systemically. The values in this protocol are called T1. A similar experiment was performed as above and found that T1 measured at week 2 following 6OHDA lesion also predicted the functional (rotometry) outcome (FIG. 5b).

At the end of the experiments (6OHDA lesioned followed by the various treatments) the striatum was stained with the pan-endothelial marker RECA-1 to visualize the state of the vasculature. All treatments had an effect (FIG. 5c). Insulin had a relatively small effect, but Dll4 significantly decreased the diameter of the vessels, suggesting an inhibition of the activation of endothelial tip cells which initiate angiogenesis (Siekmann & Lawson, Nature 445, 781-4 (2007); Hellstrom, M. et al., Nature 445, 776-80 (2007). In contrast, Ang-2 significantly increased the number of vessels, as would be expected by a pro-angiogenic cytokine (Maisonpierre, P. C. et al., Science 277, 55-60, 1997). The combined treatment (CT) did not exhibit an effect on the vasculature, either on vessel size or diameter, and did not result in bleeding, possibly due to the opposing actions of Dll4 and Ang-2. These data show that the NSC niche can be activated by treatments with varying effects on the vasculature

### Example 7

### Neuroprotection and Behavioural Recovery

To investigate the clinical potential of this treatment approach, the compounds were used in an animal model of Parkinson's disease. Adult rats were lesioned by intra-striatal 6-OH-DA (Przedborski, S. et al., Neuroscience 67, 631-47, 1995). After two weeks, a single injection of the compounds was administered in the lateral ventricle ipsilateral to the lesion. Amphetamine-induced rotations were measured bi-weekly throughout the experiment. Thirteen weeks after treatment they were given a single injection of fluorogold in the striatum to label the striato-nigral dopaminergic projection.

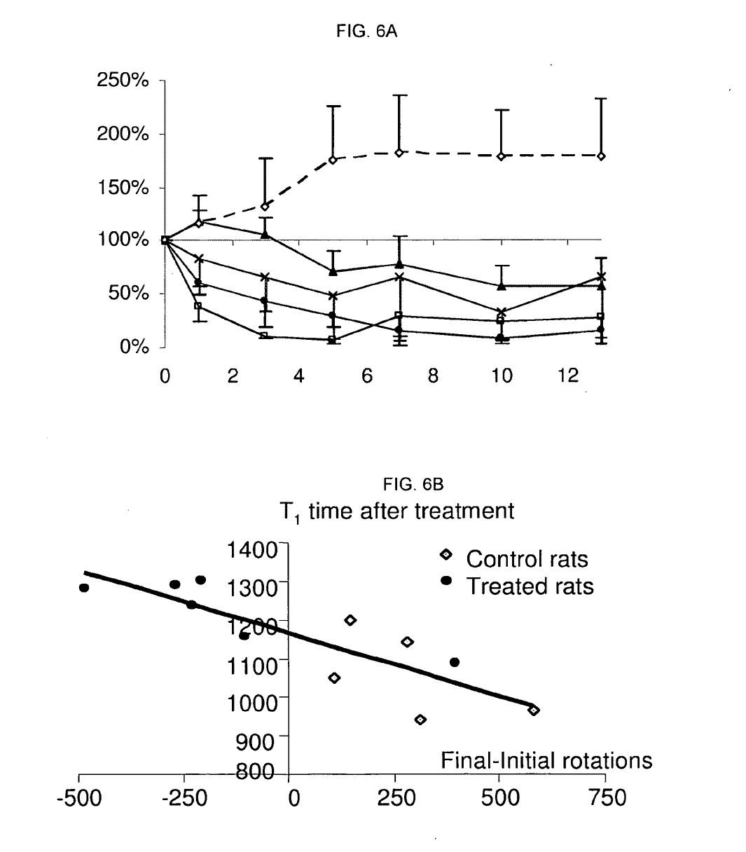

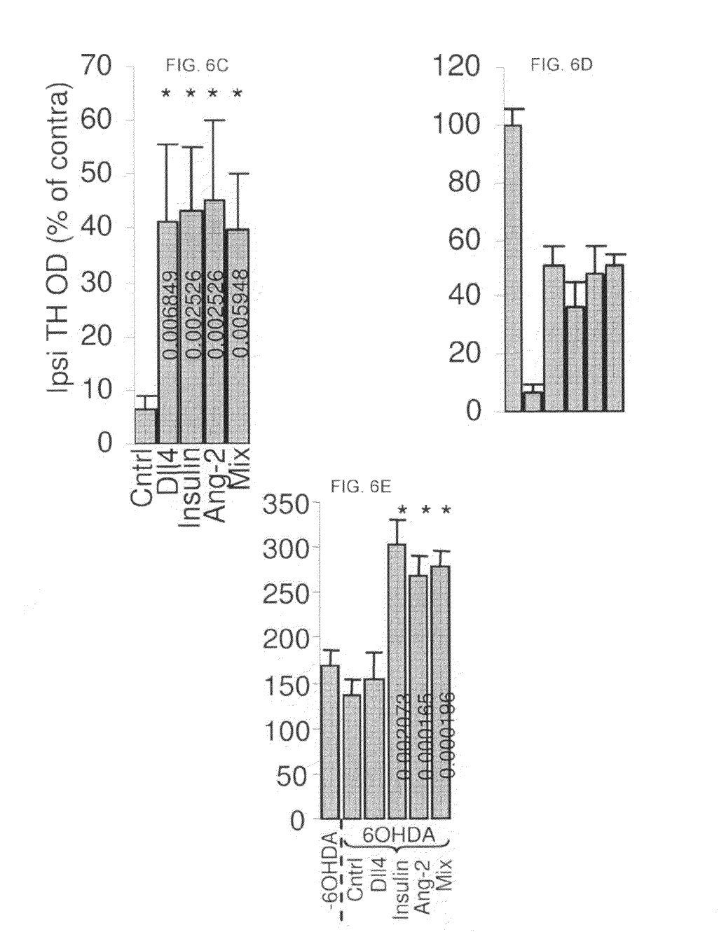

All treatments significantly reduced rotations demonstrating behavioural recovery (FIG. 6a). The behavioural improvement (expressed as the difference between rotations in the beginning and the end of the experiment) correlated with the T1 value obtained for each animal at the end of the experiment (R²=0.72, p<0.001; FIG. 6b). It also correlated with the rescue of dopaminergic neurons in the substantia nigra (number of TH+/Fluorogold+ cell bodies) (R²=0.86, p<0.001). All treatments also rescued TH immuno-reactivity in the striatum and substantia nigra and showed increased numbers of fluorogold-labelled neurons in the substantia nigra (FIGS. 6c and 6d), and TH/Fluorogold double immuno-reactivity in the substantia nigra. All treatments except for Dll4 showed increased Fluorogold+ cell body sizes in the substantia nigra, an indication of functional compensation (Russo et al., Nat Neurosci 10, 93-9, 2007) (FIG. 6e).

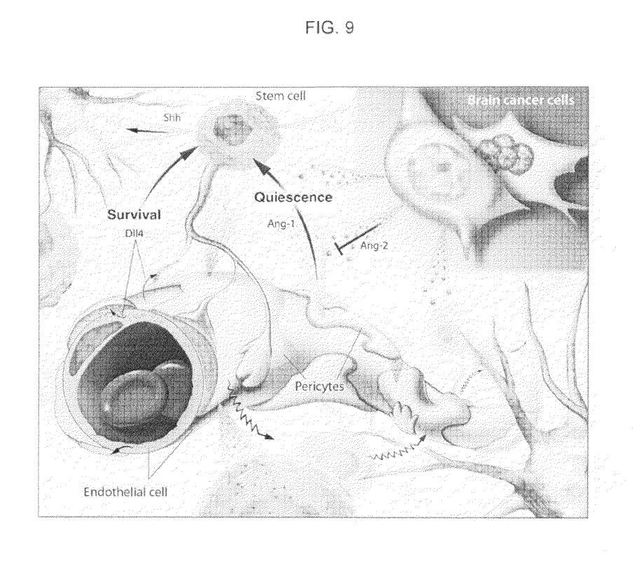

These results demonstrate the approach of considering complex interactions between cells in the niche to discover neuroprotective agents. The data provide novel pharmacological manipulations that activate the neuroprotective Akt pathway and offer a model where vascular endothelial and pericyte cells, NSC, and neurons communicate via Notch, Ang-1, Ang-2, Shh, and GDNF signals to maintain and activate the NSC niche, and protect neurons (FIG. 9).

### Example 8

### Materials and Methods for Examples 9-12

Cell Culture: E13.5 cortical embryonic mouse CNS stem cells were grown as previously described (Johe et al., Genes Dev 10, 3129, 1996). Cells were expanded in serum-free DMEM/F12 medium with N2 supplement and FGF2 (20 ng/ml) for 5 days under 5% oxygen conditions and were re-plated fresh or from frozen stocks at 1,000-10,000 cells per cm². FGF2 was included throughout our experiments, unless otherwise stated. Adult rat (3-6 months old) or adult mouse (2-4 months old) SVZ Neural Stem Cell (NSC) cultures were grown in the same medium as the fetal cultures.

Pharmacological treatments: Unless otherwise stated, we used the following concentrations: Dll4 and Ang2 (in vitro, 500 ng/ml; in vivo 12.5 μg as a single injection), FGF2 (in vitro, 20 ng/ml), JAK inhibitor (in vitro, 200 nM; in vivo, 250 ng as a single injection), insulin (in vitro, 25 μg/ml; in vivo, 40 μg as a single injection), DAPT (1 μM), Fluorogold (0.5 μg). For the survival assays, compounds were added daily and Notch ligands every 2 days for the duration of the experiment.

3D image rendering: Volocity 3D imaging software by Improvision was used on confocal z-stacks, see the improvision website.

Blood vessel counting and area quantitation: Zeiss software was used to identify, count, and measure blood vessels stained for the pan-endothelial marker RECA-1. To measure the average distance between adjacent Hes3+ cells along the same blood vessel, 25 high-power (×63) confocal z-stack images were used from brain sections stained for endothelial markers and Hes3.

In vivo Experiments: Male adult (3-6 months) Sprague Dawley rats (Charles River Laboratories), weighing 250-350 gm, was used for pharmacological treatments and lesion experiments. For 6OHDA Lesion: animals underwent unilateral lesion of the nigrostriatal dopamine pathway by stereotactical injection of 50 μg 6-hydroxydopamine (6-OHDA) into the right striatum, using the following coordinates: bregma AP +0.5 mm, ML −3.0 mm, VD +5.5 mm. For pharmacological treatments: unlesioned animals, or animals two weeks after 6OHDA lesion, were treated with different pharmacologic agents. Five μl of different drugs were stereotactically injected into the right lateral ventricle using the following stereotaxic coordinates: bregma AP −0.9 mm, ML −1.4 mm, VD +3.8 mm. The following reagents were used: Dll4 (2 mg/ml), Ang2 (1 mg/ml), insulin (8 mg/ml), Jak-Inhibitor (20 μM), either alone or in combination. After all surgeries, animals were allowed to recover from the anesthesia and were put back into their home cages, where they were given access to food and water ad libitum. For labeling of dividing cells: Animals received intraperitoneal injection of the tracer bromodeoxyuridine (BrdU) (50 mg/kg) every 12 hours for 5 days beginning on day 1 post-op to label dividing cells. Amphetamine induced rotations: At multiple time points over the course of the experiment, lesioned rats were injected intra-peritoneally with D-Amphetamine sulfate (5 mg/kg) and placed in automated rotometer bowls. Rotational behavior was assessed by monitoring total number of whole body (360°) turns over 60 min.

Immunohistochemistry: Under deep anesthesia, animals were perfused transcardially with a rinse of saline, followed by 4% formaldehyde fixative (pH 7.4). Brains were removed immediately, stored in the fixative solution overnight and then in 30% sucrose for 3 days. Brains were frozen-sectioned at 16 μm. Immunohistochemical detection of BrdU and Hes3 was performed with an antigen-retrieval step (Sections were boiled in 0.02M citrate, pH 6.0 in a microwave for 5 minutes, washed 3 times with distilled water, and incubated for 45 min in 2M HCl, at room temperature).

MRI Imaging: MnCl2 administration: Adult male Sprague-Dawley rats received stereotactical injections of 6OHDA into the right striatum, and saline injections of equal volume into the left striatum as a negative control. In order to use Mn2+ as a contrast agent (40), rats were injected intraperitoneally with 30 mg/kg MnCl2 24 hrs prior to each imaging session; during neuronal tract tracing (41), 100 mM MnCl2 was ad-ministered into the substantia Nigra 2 weeks after 6OHDA lesioning. MRI Method: MRI was performed 24 hours after the lesion, followed by weekly MRI over 7 weeks. Rats were anaesthetized with 1.5% isofluorane and placed prone in a stereotaxic holder with brain centered in a 72/25 mm volume transmit/surface receive coil ensemble. Body core temperature was maintained at 370 C with warm air flow over the rat. A pressure transducer was used to monitor the 11 respiration cycle. MR imaging was performed on a 21 cm horizontal bore 7 Tesla scanner operating on a Bruker Avance platform (Bruker Biospin Inc. Billerica, Mass.). Three mutually perpendicular slice images through the brain were acquired as scout images. Fourteen contiguous 1 mm thick axial images (slice number 6-7 centered at the Bregma) of the brain were acquired using a fast spin echo (FSE) sequence (matrix 2562, NA=8, echoes=8, TR/TE=3500/14.15 ms, Field of View [FOV]=3.2 cm); an identical set of FSE images with T1 weighting (TR/TE=355/10.25 ms) was also acquired. In order to obtain quantitative T2 values, T2 weighted axial images (TR/TE=3500/20 ms, 8 echoes, FOV=3.2 cm, matrix=1282) were acquired. A 2-D multi slice look-locker sequence (42) with 20 point samples along the inversion recovery curve was used to calculate T1 (TE/TR=2.2/104 ms, flip angle=250, FOV=3.2 cm, Matrix 128 (41), slice thickness=1 mm, inversion interval=400 ms). Quantitative T1 and T2 maps were calculated using custom written software in MATLAB (Mathworks Inc., Natick, Mass.) taking into account the perturbation caused by flip angle in determining T1.

Reagents: FGF2 (233-FB), mouse Dll4 (1389-D4), CNTF (577-NT), Fibronectin (1030-FN), human angiopoietin-2 (623-AN), from R&D; JAK Inhibitor I (420099), from Calbiochem; Polyornithine (Sigma, P-3655), ECL reagents (Pierce, 34080), polyacrylamide gradient gels (Invitrogen), BrdU (Boehringer, 84447723), Fluorogold from Fluorochrome, LLC, insulin (Sigma, 19278), Alexa-Fluor-conjugated secondary antibodies (Molecular Probes), HRP-conjugated secondary antibodies (Jackson Immunoresearch), DAPI (Sigma, D-8417), and general chemicals from Sigma. For immunohistochemical staining and Western blotting, antibodies against the following markers were used: nestin (Chemicon, MAB353), Tuj1 (Covance, MMS-435P), GFAP (Dako, z0334 and Chemicon, MAB360), CNPase (Chemicon, MAB326), BrdU (Accurate, H5903), Sox2 (R&D, MAB2018); pSer473-Akt12 (92715), pThr308-Akt (9275), pSer2448-mTOR (29715), from Cell Signaling; STAT3 (482), pSer727-STAT3 (8001-R), pTyr705-STAT3 (7993), Akt (5298), Hes3 (25393), Ang1 (sc-6319), Ang2 (sc-7015), Tie-2 (sc-324), Tie-2 (sc-31266), Shh (sc-1194) from Santa Cruz; α-tubulin (Sigma, T-6074); tyrosine hydroxylase (P80101 and P40101 from Pel-Freez); RECA-1 (MCA 970GA) from Serotec; pTie-2 (AF2720) from RnD Systems.

Statistical analysis: results shown are the mean±s.d. or mean±s.e.m. as indicated. Asterisks identify experimental groups that were significantly different (p-value, 0.05) from control groups by the Student's t-test (Microsoft Excel).

### Example 9

### Tie-2 Receptor is Expressed in the SVZ and Blood Vessels of the Rat Brain

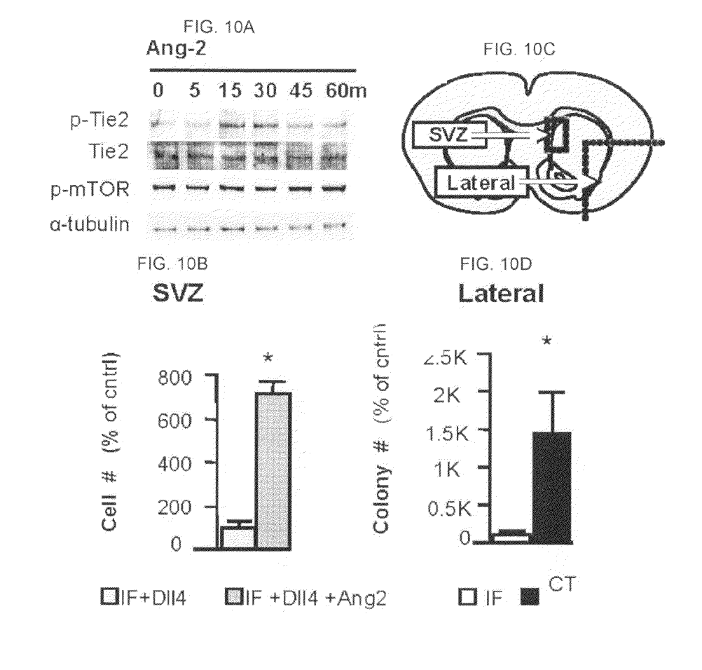

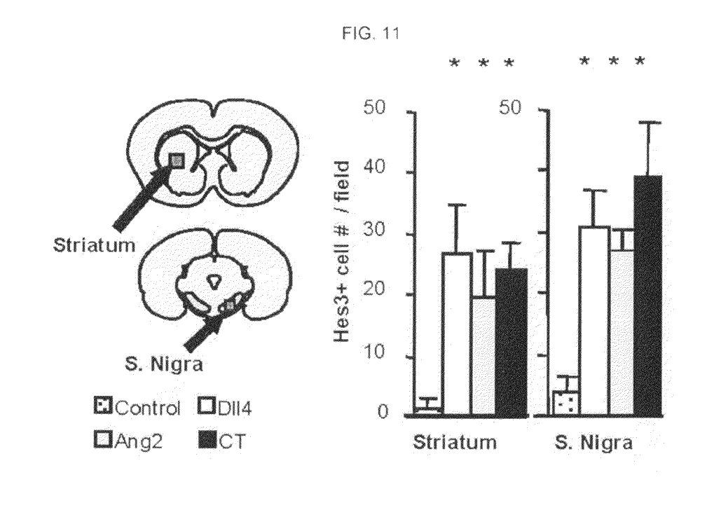

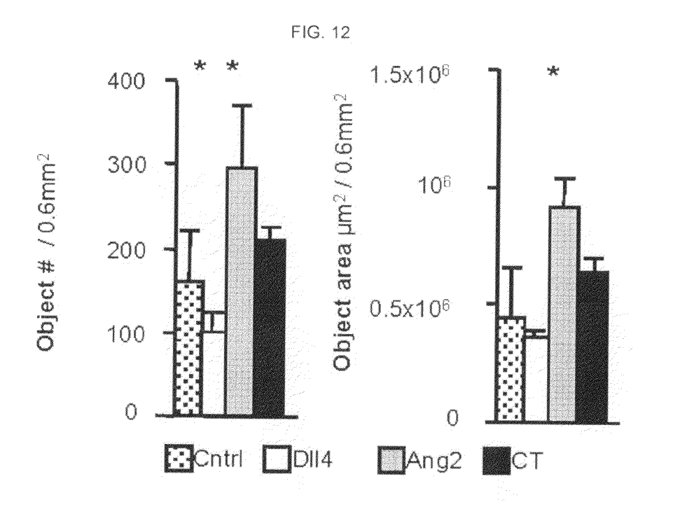

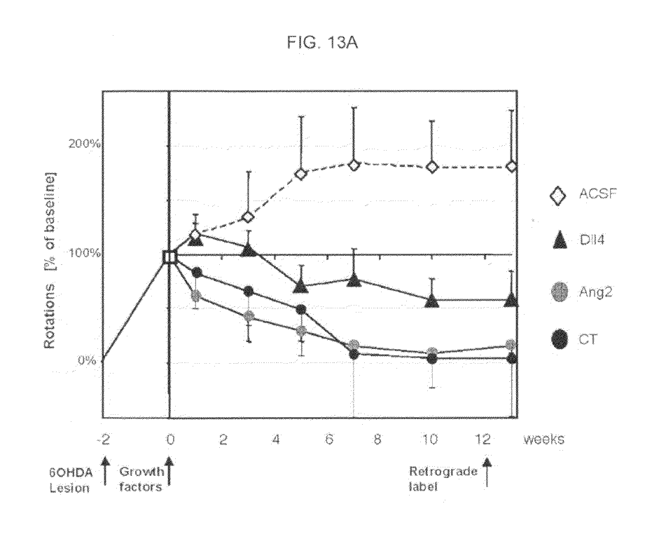

Three characterized Tie-2 receptor antibodies were used to determine if the Tie-2 receptor was expressed in the immature neural precursor cells found in the SVZ of the adult brain. When ligand is bound, the Tie-2 receptor becomes phosphorylated. An antibody that recognizes the activated receptor (RnD Systems, AF2720) and another antibody that binds to a distinct site (Santa Cruz, sc-324) identified many positive cells in the subventricular zone of the adult rat brain. In regions that were more distant from the ventricle, Tie-2 was expressed predominantly on blood vessels and endothelial cells reacted with antibodies against the activated receptor. Precursor cells that proliferate in vitro can be readily derived from the fetal and adult brain. The homogeneity of these cell populations in attached culture and their potential to generate neurons and glia is supported by clonal analysis. Anti-Tie-2 antibodies (anti-C-terminus: Santa Cruz, sc-324; anti-N terminus: Santa Cruz, sc-31266) bound to the vast majority of undifferentiated neural precursors and, consistent with the in vivo data, this reactivity was rapidly lost when the precursor cells differentiate. Western blots showed that exogenous Ang2 promoted phosphorylation of Tie-2 with no effect on mTOR, a serine-threonine kinase with an important role in growth (FIG. 10a). These data illustrate that neural precursors express a functional Tie-2 receptor. The Notch ligand Dll4 co-operates with insulin (I) and basic Fibroblast Growth Factor (FGF2) to promote efficient ex-vivo growth of fetal and adult neural precursor cells. Ang2 enhanced these effects, to give a 7-fold increase in the numbers of proliferating cells when cells derived from the adult SVZ were placed in culture (FIG. 10b; p for SVZ=2.15×10⁻⁵). Because of this increased efficiency, studies were performed to determine if proliferating neural precursors could be obtained from lateral regions of the forebrain distant from the SVZ. FIG. 10c provides a schematic representation of the areas dissected from the adult rat brain (SVZ, Lateral: ˜bregma 1.5 to −0.36 mm. Astrocytic differentiation can be driven by the JAK/STAT pathway. Jak kinase inhibitors can promote the ex-vivo proliferation of neural stem cells, pancreatic precursors and human embryonic stem cells. When a Jak inhibitor was included with Dll4, insulin, FGF2 and Ang2 (combination treatment, CT), a 14-fold increase in colonies of proliferating cells was obtained when cells dissociated from the lateral region of the forebrain were placed in culture (FIG. 10d; lateral p=0.036686). This colony number assay measured the survival of precursor cells that could differentiate into neurons and glia (See, Table 1). These results show that Ang2 contributes to the growth of multipotent neural precursors in vitro.

TABLE 1
Differentiation potential of adult SVZ and parenchymal
precursors. Ratios of neurons (TUJ1+), astrocytes
(GFAP+), and oligodendrocytes (CNPase+) following
Notch activation (7-d Notch + FGF2, 10-d withdrawal).

| | % Neurons | % Glia | % Oligodendrocytes |
|---|---|---|---|
| | (TUJ1) | (GFAP) | (CNPase) |
| SVZ | 43 ± 10 | 51 ± 10 | 9 ± 2 |
| Parenchymal | 37 ± 7 | 32 ± 4 | 30 ± 3 |

### Example 10

### Angiogenic Factors Activate Neural Precursors In Vivo

The Hes and Hey genes encode bHLH transcription factors that mediate transcriptional response to Notch activation. Genetic approaches in the mouse show that Hes3 interacts with other Hes genes to control neuroepithelial cells in the developing brain. In neural precursors, Hes3 mRNA levels are controlled by a γ-secretase dependent cleavage of the Notch receptor and, in turn, Hes3 is responsible for sustained expression of sonic hedgehog (Shh; Androutsellis-Theotokis et al., Nature. 442, pp. 823-6, 2006). Shh is a secreted factor capable of promoting both neuroepithelial precursors and vascular development where it stimulates expression of angiopoietins. An antibody against Hes3 recognized precursors located in the SVZ and cells throughout the adult brain that extended short processes that contact blood vessels identified by the endothelial specific antigen, RECA-1. The SRY transcription factor Sox2 plays a role in establishing the pluripotent state of cells in the early embryo and is also required for neurectodermal differentiation. As illustrated in FIG. 11, a 3D rendering of confocal optical sections clearly showed that most Hes3+ cells contain a Sox2+ nucleus. In the adult, 12% of the Sox2+ cells in the striatum (12±4%; n=4) and 10% of the Sox2+ cells in the substantia nigra co-expressed Hes3 (10±3%; n=4). Almost all (97.2%±6.8 in striatum; 95.2%±11.6 in S. Nigra) the Hes3+ cells co-expressed Sox2. Hes3+ cells co-expressed GFAP but they were only a small subset of the Hes3+ cells in the adult brain. Consistent with the link between Hes3 and Shh in vitro, almost all the Hes3+ cells co-expressed Shh (97.6%±5.8 in striatum; 95.1%±7.6 in S. Nigra). The rapid response of Hes3 to growth factor activation in vitro, suggests that the number of Hes3+ cells could be a sensitive measure of growth factor responses over large areas of the brain.

Single ventricular injections of the Notch ligand Dll4 also rapidly increased the numbers of Hes3+ and Tie-2+ precursor cells in the SVZ. Relative to controls that contained bovine serum albumin (BSA), an increase in the numbers of Hes3+ precursors was found in all brain regions including the spinal cord following a single ventricular injection of growth factors. The ventral areas of the fore- and mid-brain contain the axons and five cell bodies of dopamine neurons that can respond to these treatments. In the striatum and in the substantia nigra, a greater than 10-fold increase in Hes3+ cells was seen 5 days after treatment with the three conditions that supported neural precursors in vitro (FIG. 11; the regions assessed are marked by squares). Significant increases in Hes3+/BrdU+ double-labeled cells were also observed in the striatum and midbrain in treated animals. These results show that a single injection of angiogenic factors activates precursor cells that are widely distributed in the brain.

### Example 11

### Combination of Pro- and Anti-Angiogenic Factors Maintain Normal Vascular Density

The results described above demonstrate that a single injection of angiogenic factors activates precursor cells that are widely distributed in the brain. As this activation is achieved with growth factors known to have angiogenic effects, these treatments were evaluated on the structure of the vascular system. The endothelial specific antigen RECA-1 was used to measure the number and size of blood vessels in the striatum 13 weeks after a single treatment with growth factors (FIGS. 14a and b). Using pattern recognition software to determine the number and size of blood vessels, Dll4 and Ang2 were demonstrated to have opposing and long-lasting effects on the vascular system. In contrast, the CT treatment did not cause a net change in the number or size of blood vessels (FIGS. 14a and b). This result suggests that the balance between pro- and anti-angiogenic signals blocks a large effect on the vascular system while allowing the activation of endogenous neural precursor cells.

### Example 12

### Injured Dopamine Neurons are Protected from Death by Single Treatments with Angiogenic Factors

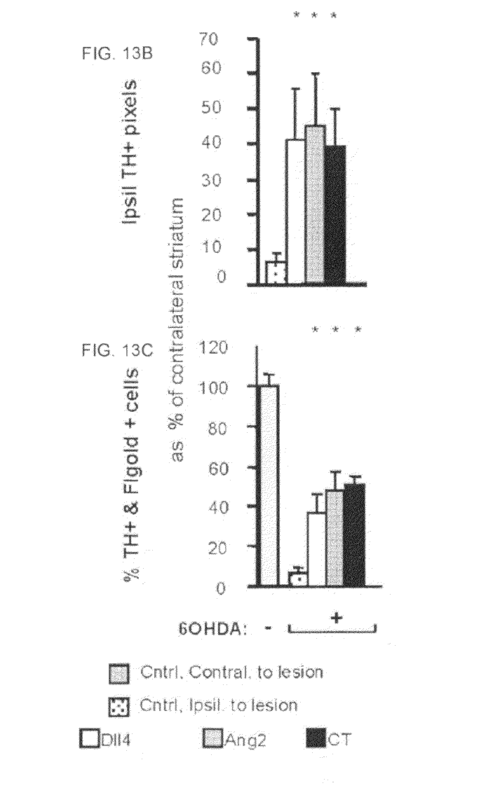

The growth factor delivery to the ventricle stimulates rapid responses in brain regions that contain the cell bodies and axons of dopamine neurons. To determine if these treatments support the survival of dopamine neurons, an established model was used where unilateral delivery of 6OHDA in the striatum leads to the progressive death of dopaminergic neurons that can be monitored by a simple behavior (Ungerstedt, Acta physiologica Scandinavica Supplementum 367: 49-68, 1997; Przedborski et al., in Neuroscience 67: 631-47, 1995). Rats move in response to amphetamine stimulation of dopamine release and in unilaterally lesioned animals, a bias in movement away from the lesion provides a simple measure of dopamine output in the two sides of the brain. At 2 weeks after the lesion, approximately half the dopamine neurons were already irretrievably injured and these animals showed a marked rotational bias that became progressively worse as the remaining dopamine neurons were lost (FIG. 13a). At this mid-point in neuron loss, three groups of lesioned animals were given a single intraventricular injection of angiogenic factors and the control group was injected with BSA. The rotational behavior measured over the next 13 weeks demonstrated that all three treated groups showed recovery in the rotation assay (FIG. 13a; n=4-7 for each group). The Dll4 treated animals showed only partial recovery, while the Ang2 and CT treated animals showed a complete and stable recovery in rotational bias. The distribution of dopaminergic axons in the striatum was assessed by immunohistochemistry for tyrosine hydroxylase (TH). At 13 weeks, in the absence of angiogenic treatment, there were few TH+ processes in the lesioned striatum. In contrast, in the treated animals, TH expression in the striatum was sustained at 40% of the levels in the uninjured striatum in all the treated groups (FIG. 13b). One week prior to the end of the study, a retrograde fluorescent tracer was injected into the ipsi- and contra-lateral striatum. These data showed that less than 10% of the dopamine neurons were present in the controls that received a CSF injection. In contrast, treated animals retained 40% of the number of dopamine neurons (FIG. 13c). Nissl staining also confirmed the presence of neuronal cell bodies in the substantia nigra of treated animals. When BrdU was given for three days following the treatment, no BrdU+/TH+ cells were seen in the substantia nigra. The behavioral recovery, TH expression in the striatum and the numbers of retrogradely labeled cell bodies in the substantia nigra provides three measures of the rescue of injured dopamine neurons. These results demonstrate the ability of angiogenic factors to rescue injured dopmine neurons. However, hemorrhage and an enlarged ventricle associated with the 6OHDA lesion was seen in animals treated with Ang2, this pathology was not observed with other treatments.

To determine if there were changes in the permeability of the blood brain barrier during the treatment period, magnetic resonance imaging (MRI) with gadolinium chelate was used to monitor the integrity of the vascular system in the ispi- and contra-lateral striatum following the 6OHDA lesion. This contrast agent is routinely used to visualize blood brain barrier disruption but no signal was detected in the striatum of lesioned animals. Manganese enhanced MRI (MEMRI) is sensitive to small changes in blood brain barrier permeability, neuronal activity or connectivity. A shortening of the T₁-weighted Mn₂₊ signal became prominent in the central region of the ipsilateral striatum in the 3 weeks following the lesion and was subsequently sustained in untreated animals. In contrast, elevated Mn₂₊ uptake was not observed in the T₁ image of treated animals measured at 15 weeks after lesion (n=10, R₂=0.72, p<0.001). These data show that the rescue of dopamine neurons is accomplished without major changes in vascular permeability.

Previous studies show that signals from endothelial cells support precursors in the central nervous system. The results presented herein show that a single injection of angiogenic growth factors activates neural precursors across wide areas of the adult brain and rescues midbrain dopamine neurons from a lethal injury. Grafts of dopamine neurons derived from mouse or human embryonic stem cells provide evidence for the potential of cell replacement approaches to this disease. The reprogramming of adult dopamine neurons can be generated from induced pluripotent (iPS) cells. The data presented herein provide a strategy to stimulate widespread regenerative responses that may complement or replace therapeutic approaches focused on specific types of neuron.

The data show that four treatments increase the number of stem cells/precursors in the adult brain and exert neuroprotective effects on neurons: Ang-2, Delta-4, insulin, and the combination treatment (“CT”, which includes Ang-2, Delta-4, insulin, and a JAK kinase inhibitor). Of these treatments, the individual agents can have some undesirable effects: Ang-2 also increases the number and size of blood vessels and increases the risk of bleeding; Delta-4 reduces the number and size of blood vessels; insulin increases the number of blood vessels adjacent to the injection site. The CT, however, increased the numbers of stem cells/precursor cells, induced neuro-protection, and maintained the normal vascular state (i.e. it shows no net change in blood vessel numbers or size). This unexpected result that pro-angiogenic and anti-angiogenic factors can be used in combination (as in the CT) to retain their ability to increase stem cell/precursor numbers and protect neurons from death, but cancel out their effects on the vasculature provides unique therapeutic strategies. Without being bound by theory, the data suggest that degenerative diseases do not focus on a single cell, but incorporate multiple cell types and affect the whole architecture of the brain.

### Example 13

### Tie-2 Expression and the Absence of Cholera Toxin Binding Identify Stem Cells

In neural stem cell (NSC) cultures, expression of Tie-2 characterizes actively proliferating stem cells. The Cholera toxin (Ctx) B subunit is non-toxic and binds to lipid rafts; it is commercially available conjugated to a variety of probes. The Tie-2 negative cells can be recognized because they bind the cholera toxin subunit that interacts with lipid rafts. These Tie-2⁻ and Ctx⁺ cells have distinct proliferative properties.

TABLE 2
Expression of Tie-2 in NSC cultures

| | Tie-2 Antibody signal | | |
|---|---|---|---|
| | Phospho-Tie-2 | Tie-2 (324) | Tie-2 (31266) |
| Stem cells | HIGH | ~2% | HIGH |
| Differentiating cells | LOW | 0% | MEDIUM |

The Cholera toxin B subunit (conjugated to a fluorophore) was also used to assess the presence of lipid rafts in NSC cultures

TABLE 3
Presence of Lipid Rafts

| | Cholera toxin B subunit binding |
|---|---|
| Stem cells | LOW |
| Differentiating cells | HIGH |

A protrusion of the cell membrane was identified in neuronal stem cells (NSCs) when the Tie-2 receptor is concentrated. This can be visualized using antibodies that bind Tie-2. The protrusion was termed a spike. These spikes appear 30 minutes after treatment with pharmaceutical agents. D!!4 and Nng-2 increase spike number thirty minutes after treatment. Ang-1 and CNTF, which do not promote stem cell survival, do not increase spike number. Thus, a novel sub-cellular structure ahs been identified. These structures could regulate the responsiveness of cells to treatments.

TABLE 4
Incidence of spikes in NSC cultures

| | % of cells with at least one spike |
|---|---|
| Control | 1.2 |
| Dll4 | 2.1 |
| Ang1 | 0.8 |
| Ang2 | 2.5 |
| CNTF | 0.8 |
| Cocktail | 2.4 |
| Differentiated cells | 0 |

Thus, a novel protrusion of the cell membrane in NSCs has been identified where the Tie-2 receptor is concentrated. This can be easily visualized using an antibody against Tie-2 that recognizes a particular epitope. Only a fraction (˜2%) of NSCs show this protrusion (for now, we call it the “spike”), suggesting that it is a transient compartment. Indeed, the spike is devoid of actin filaments or tubulin. The spikes can be detected within thirty minutes of contacting the cultures with an the factors. Thus, the spikes provide a rapid assay for the effect of pharmacologic agents.

It will be apparent that the precise details of the methods or compositions described may be varied or modified without departing from the spirit of the described invention. We claim all such modifications and variations that fall within the scope and spirit of the claims below.

1. A method for increasing the number of stem cells, precursor cells or both, comprising contacting the stem cells or precursor cells with an effective amount of a Notch ligand, a therapeutically effective amount of a growth factor and a therapeutically effective amount of angiopoietin-2, thereby increasing the number of stem cells, precursor cells, or both.

2. The method of claim 1, wherein the method is a method for increasing the number of stem cells.

3. The method of claim 2, wherein the stem cells are neuronal stem cells.

4. The method of claim 1, further comprising contacting the cells with a therapeutically effective amount of a Jak inhibitor.

5. The method of claim 1, wherein the Notch ligand is Delta.

6. The method of claim 1, wherein the growth factor is a glial derived neurotrophic factor (GDNF).

7. The method of claim 1, wherein the growth factor is a fibroblast growth factor.

8. The method of claim 7, wherein the fibroblast growth factor is fibroblast growth factor (FGF)-2.

9. The method of claim 1, wherein the growth factor is insulin.

10. The method of claim 1, wherein the growth factor is insulin and the Notch ligand is Delta.

11. The method of claim 1, wherein the stem cells or precursor cells are in vivo.

12. The method of claim 11, wherein the method is a method for increasing the number of precursor cells, and wherein the precursor cells are neuronal precursor cells.

13. The method of claim 1, wherein the stem cells or precursor cells are in vitro.

14. A method for treating a neurodegenerative disorder or a spinal cord injury in a subject, comprising
selecting a subject with a neurodegenerative disorder or a spinal cord injury; and
administering to the subject a therapeutically effective amount of a growth factor, a therapeutically effective amount of angiopoietin-2 and a therapeutically effective amount of a Notch ligand,
thereby treating the neurodegenerative disease or the spinal cord injury in the subject.

15. The method of claim 14, wherein the Notch ligand is Delta.

16. The method of claim 14, wherein the growth factor is insulin.

17. the method of claim 14, wherein the growth factor is glial derived neurotrophic factor.

18. The method of claim 14, wherein the growth factor is a fibroblast growth factor.

19. The method of claim 18, wherein the fibroblast growth factor is fibroblast growth factor (FGF)-2.

20. The method of claim 14, further comprising administering to the subject a therapeutically effective amount of a Jak inhibitor.

21. The method of claim 14, wherein the therapeutically effective amount of the growth factor, the therapeutically effective amount of angiopoietin-2 and the therapeutically effective amount of the Notch ligand are administered locally.

22. The method of claim 14, wherein the therapeutically effective amount of the growth factor, the therapeutically effective amount of angiopoietin-2 and the therapeutically effective amount of the Notch ligand are administered intraventricularly in the brain.

23. The method of claim 14, wherein the neurodegenerative disease is Parkinson's disease.

24. The method of claim 14, wherein the neurodegenerative disease is Alzheimer's disease.

25. The method of claim 14, wherein administering comprises delivering a single pulsatile dose of the growth factor, angiopoietin-2 and the Notch ligand.

26. The method of claim 14, wherein the subject has a spinal chord injury.

27. The method of claim 14, wherein administering the therapeutically effective amount of the growth factor, the therapeutically effective amount of angiopoietin-2 and the therapeutically effective amount of the Notch ligand results in an increase in the number of cells that express Hairy enhancer of split (Hes3) in the subject.

28. The method of claim 22, wherein the growth factor is insulin and the Notch ligand is delta.

29. The method of claim 28, wherein administering the therapeutically effective amount of the growth factor, the therapeutically effective amount of angiopoietin-2 and the therapeutically effective amount of the Notch ligand results in an increase in the number of cells that express Hairy enhancer of split (Hes3) in the subject.
